(19) United States
(12) Patent Application Publication
(10) Pub. No.: US 2026/0249241 A1
(43) Pub. Date: Aug. 27, 2026

(54) METHOD OF SEQUESTERING ATMOSPHERIC CARBON

(71) Applicant: FABRICNANO LIMITED, London (GB)

(72) Inventors: Grant Aarons, London Greater London (GB); Jonathan Burns, London Greater London (GB); Vishnu Srinivasamurthy, London (GB)

(21) Appl. No.: 19/123,309

(22) PCT Filed: Apr. 2, 2024
(86) PCT No.: PCT/GB2024/050905

§ 371 Date: Apr. 22, 2025

(30) Foreign Application Priority Data
Mar. 30, 2023 (GB) 2304769.9
Dec. 27, 2023 (GB) 2320054.6

(51) Int. Cl. B01D 53/85; B01D 53/62

(52) U.S. Cl. CPC B01D 53/85; B01D 53/62; B01D 2255/804; B01D 2257/504

(57) ABSTRACT

This invention relates to carbon sequestration, in particular by enhanced weathering. The methods generally utilise an enzyme, typically carbonic anhydrase. In particular, the invention relates to methods of sequestering atmospheric carbon by applying Carbonic Anhydrase, and optionally rock, to soil or earth. An immobilised enzyme is also provided, along with the use of that enzyme to sequester atmospheric carbon. Mixtures comprising carbonic anhydrase are also provided, for example carbonic anhydrase mixed with one or more of soil, seed and rock.

### FIELD OF THE INVENTION

This invention relates to carbon sequestration, in particular by enhanced weathering. In particular, the invention relates to methods of sequestering atmospheric carbon by applying Carbonic Anhydrase, and optionally rock, to soil or earth.

### BACKGROUND OF THE INVENTION

Enhanced weathering sequesters carbon from carbon dioxide, accelerating the natural process of rock weathering. The application of finely-ground rock to agricultural land is one known method of utilising enhanced weathering to sequester atmospheric carbon.

de Oliveira Maciel et al. (Chemosphere, July 2022, 299, 134419, 1-25) review the use of Carbonic Anhydrase to boost carbon dioxide sequestration. The authors note that Carbonic Anhydrase can be utilised in accelerated weathering. Ren et al. (Colloids and Surfaces B: Bioterfaces, August 2021, 204, 111779, 1-10) review immobilisation of Carbonic Anhydrase and note the high cost of immobilisation supports.

There remains a need to improve methods of carbon sequestration, to address anthropogenic climate change and increase food security.

### SUMMARY OF THE INVENTION

The present disclosure relates in general to methods of carbon sequestration by enhanced weathering. The methods generally utilise an enzyme, typically carbonic anhydrase. In particular, the methods relate to application of an enzyme (such as carbonic anhydrase) and rock to soil, earth or ground. The methods also relate to contacting a plant or plant seed with Carbonic Anhydrase and then contacting that enzyme-inoculated plant material with the soil, i.e. by planting the seed or plant into the soil. Typically, this is carried out at large scale such a planting one or more fields with enzyme-inoculated seeds or plant material, or re-foresting an area with enzyme-inoculated tree roots.

This application of enzyme to a typically large area differs from “smokestack” carbon sequestration where carbon dioxide is removed from a chimney and where enzyme re-use and recycling is typically required.

The enzyme may be any enzyme that is capable of enhancing carbon sequestration into soil. The enzyme may be any enzyme that is capable of increasing plant yield.

The enzyme may be any enzyme that is capable of capturing ambient carbon dioxide.

The enzyme is typically a carbonic anhydrase. Carbonic anhydrases are enzymes that convert carbon dioxide and water into bicarbonate and protons.

Enhanced weathering aims to accelerate the natural weathering of rock by spreading finely ground silicate rock, such as basalt, onto surfaces which speeds up chemical reactions between rocks, water, and air. It removes carbon dioxide (CO₂) from the atmosphere, permanently storing it in solid carbonate minerals.

Enhanced weathering technology typically uses rock. This could include Olivine rock, Basalt, and other rocks rich in metal ions. The rock may be particulate.

Enhanced weathering projects have recently had MRV (Measurement Reporting, Verification) models accepted for carbon credits that can be sold commercially. Carbon credits are typically sold as carbon stored on a 20 year horizon. The MRV model is built around these timescales. Carbon sold at this price is currently charged at $200/ton of carbon sequestered from the ambient air.

Carbon credit duration can be securitized such that 1 year carbon sells for $800/ton and 20 year carbon sells for up to $200/ton. These prices come from present day demand shocks/high levels always being greater than prolonged carbon purchasing. If carbon sequestration projects could be accelerated than the total value from any project would be multiplied by 2×, 3×, 4×, or more.

Carbonic anhydrase is an enzyme that is widely known to produce carbonic acid from ambient CO₂ and H₂O. Carbonic Anhydrase is readily available at scale, studied extensively because of its prominence in natural systems as a pH regulator, and many commercial projects exist to try and sequester carbon using this enzyme.

Carbonic anhydrase can sometimes be very stable, but this depends on the host organism and enzymes from extremophile host organisms typically exhibit the highest stability to prolonged and continuous deactivation modes.

The Carbonic Anhydrase is typically immobilised to a solid support. The solid support may comprise or consist of rock, for example particulate rock such as basalt particles. The solid support may be a solid support that is separate from the rock, and which can be mixed with the rock.

In some embodiments, the solid support can be the rock, for example the particulate rock or rock sand. Accordingly, the disclosure provides immobilizing directly onto the ground rocks, e.g. finely ground rocks, via any binding chemistry and/or interaction. In some embodiments, the Carbonic Anhydrase is immobilised to the rock, optionally directly to the rock, optionally by covalent bond or by non-covalent means such as physical adsorption.

In some embodiments, cheap materials are used as the solid support for immobilisation to ensure commercial viability (e.g. waste solid supports and materials <$10, beneficially <$5, further beneficially <$1 kg, further beneficially <$0.5/kg). Examples of such materials include corn husks, waste cellulose, waste fibrous materials, typically farming additives used for liming such as vermiculite.

In some embodiments, the solid support is a plant seed.

In some embodiments the disclosure provides soil+finely ground rock+enzyme+/−immobilization.

In some embodiments the Carbonic Anhydrase enzyme is not immobilised, i.e. is a “free” enzyme. In some embodiments, the free enzyme binds to plant roots in the top soil or plant material above the top soil.

In some embodiments, the Carbonic Anhydrase enzyme is designed or engineered to accomplish retention at a specified soil depth with or without immobilisation, for example artificially making larger enzyme radius of gyration, and/or making enzyme charged so that it binds to plant roots at the optimal depth where CO₂ is typically present at the roots. Accordingly, in some embodiments, the Carbonic Anhydrase enzyme is immobilised to the plant root.

Carbon can be introduced into the soil by the fixing of atmospheric carbon dioxide into plant biomass. The carbon in the plant biomass may then be converted into carbon dioxide by the respiration of organisms that aid the decomposition of the dead plant matter. Therefore, immobilising the enzyme to the plant root may be particularly beneficial, as this may bring the Carbonic Anhydrase enzyme in closer proximity to the organisms that aid the decomposition of the dead plant. In some embodiments, therefore, the solid support is a plant root or other part of a plant.

Carbon sequestration via enhanced weathering technology is readily scalable and almost profitable, but too slow to be economically attractive and impactful. The entire commercial value of enhanced weathering lies at the intersection of scale and speed. Scale is easy, speed has been elusive and comes with costs (e.g. grinding rock to be smaller and faster to react with naturally available carbonic acid, but the level of carbonic acid in soil is relatively low concentration and thus limiting on the rate of carbon sequestration).

In some embodiments, an increase in soil organic carbon comprises an increase in the level of stable carbon in the soil.

In some embodiments, an increase in soil organic carbon comprises an increase in aggregate stability of the soil.

Typically, the “finely” ground rock particle sizes used in enhanced weathering are on the order of greater than 1 mm or even greater than 10 mm in diameter whereas enzymes are on the order of less than 10 nm in diameter. Enzymes are therefore much smaller, and as in reactor design around immobilized enzymes, this means that the enzyme is likely to leave the reaction environment before any chemical production is accomplished.

In some embodiments, the rock is particulate and has an average particle size of 50 micrometres or more, more typically 300 micrometres or more for example 500 micrometres or more. In some embodiments, the rock is particulate and has an average particle size of 5 millimetres or less. In some embodiments, the rock is particulate and has an average particle size of 4 millimetres or less. In some embodiments, the rock is particulate and has an average particle size of about 500 micrometres to about 5 millimetres micrometres. In some embodiments, the rock is particulate and has an average particle size of about 50 micrometres to about 4000 micrometres. In some embodiments, the rock is particulate and has an average particle size of about 500 micrometres to about 4000 micrometres. In some embodiments, the rock is particulate and has an average particle size of about 500 micrometres to about 2000 micrometres, or about 500 micrometres to about 1500 micrometres, or about 500 micrometres to about 1000 micrometres, or about 500 micrometres to about 750 micrometres. In some embodiments, the rock is particulate and has an average particle size of about 1 millimetre to about 4 millimetres, or about 2 millimetres to about 4 millimetres, or about 750 micrometres to about 4 millimetres, or about 750 micrometres to about 4.5 millimetres. In all of these embodiments, a typical rock is basalt.

In an improved method of enhanced weathering described herein, the enzyme stays close to the finely ground rock to help accelerate the carbon sequestration through local carbonic acid formation. The disclosed methods and compositions therefore resist the enzyme from being pushed deeper and deeper into the soil.

Enhanced weathering works effectively when ambient CO₂ is brought down to the soil (or ground, or earth) by natural rainfall, although this could be extended artificially through techniques such as irrigation fields. When it rains and H₂O brings CO₂ to the Carbonic Anhydrase enzyme, carbonic acid forms, but also the enzyme tends to leach deeper into the soil because the enzyme is much smaller than the soil particle size and enzyme packing/residence time in the soil amongst the finely ground rock is vanishingly short. This drives the whole application of enzymes to the enhanced weathering process to be technoeconomically unviable.

By increasing the surface area and particle size of the enzyme through immobilization onto a solid support biocatalyst, then the enzyme can be applied in economically efficient dosage to any enhanced weathering process and accelerate the carbon sequestration through prolonged proximity to the finely ground rock with charged metal ions.

Methods of the present invention are particularly advantageous as they utilise Carbonic Anhydrase in enhanced weathering applications to accelerate the ability of finely ground rocks to sequester carbon. The application of enzymes to finely ground rock for the purpose of enhanced weathering and carbon sequestration is beneficial. Advantageously, methods of the present invention bring about faster climate change impact from the same footprint and faster soil health recovery. Soil health may comprise increasing levels of organic soil matter, changing pH, and/or changing microbial growth patterns.

Certain aspects of the invention comprise one or more, for example all, of the following features:
1. An enzyme is selected to improve the enhanced weathering technology (in one of many technical and commercial ways).
   a. Specifically, the Carbonic Anhydrase enzyme rapidly accelerates the formation of carbonic acid in the soil and this is rapidly interacted with metal ions, optionally provided by the finely ground rock to accelerate the overall enhanced weathering. The metal ions may already be present in the soil.
2. A solid support or solid support material may be selected for compatibility with the end field of use. The solid support may be a seed or a plant, or a rock.
3. A binding chemistry or interaction is chosen for linking the enzyme to the solid support or solid support material. This can also selected for compatibility with the end field of use.
4. The enzyme may be immobilized on the solid support or solid support material before application to the field of use.
5. The enzyme can be pre-selected for compatibility with the solid support or solid support material and binding chemistry and/or interaction chosen.
6. The immobilized enzyme is stabilized to prolonged continuous deactivation modes typically experienced for free enzymes (e.g. thermal).
7. The immobilized enzyme is shielded from any deactivation and/or degradation due to protease activity because it is fastened to a much larger entity and restricting the protease from acting on the enzyme of interest.
8. The immobilized enzyme is retained in the top soil being exposed to enhanced weathering technology (e.g. finely ground rock) due to the physical properties of the solid support (e.g. larger geometric size, charge, density)
9. The solid support or solid support material is chosen to improve the top soil retention of the immobilized enzyme.
10. The solid support or solid support material is chosen to promote the immobilized enzyme concentration/presence at a specified depth in the soil (e.g. might be nearer the roots of the natural plant organic matter).
11. The solid support or solid support material is chosen to promote proximity to some element of the enhanced weathering process (e.g. finely ground rock, top soil plant organic matter, or anything else that might be deployed with the enhanced weathering technology)

In some aspects, natural (and/or unnatural) acids (and/or bases) can be created naturally (and/or unnaturally) in situ and react with the charged cation, a divalent metal cation and/or a monoatomic dication.

### BRIEF DESCRIPTION OF THE DRAWINGS

FIG. 1A shows optical density readings from a 4-nitrophenyl acetate (4-NPA) assay performed using Carbonic Anhydrase (CA) obtained from bovine blood. The blood was obtained from Amazon steak meat.

FIG. 1B shows the product titre obtained from a 4-nitrophenyl acetate (4-NPA) assay performed using CA obtained from Amazon steak, Sainsbury's steak, or pasteurised milk.

FIG. 2A shows a plot of activity slopes of CA obtained commercially, bovine serum, blood obtained from Amazon steak, Sainsbury's steak, or pasteurised milk.

FIG. 2B shows an activity kinetic plot of the buffer used in FIG. 2A only, as negative control.

FIG. 2C shows an activity kinetic plot for CA at various dilutions.

FIG. 2D shows an activity kinetic plot for CA obtained from blood obtained from Amazon steak meat.

FIG. 3 shows a bar graph showing relative activity of commercially-obtained CA and CA obtained from blood obtained from Amazon steak meant at various temperatures.

FIG. 4 shows the product titre obtained from a 4-nitrophenyl acetate (4-NPA) assay performed using commercially-obtained CA or CA obtained from blood obtained from Amazon steak meat. Each CA was immobilised to different solid supports, namely sand, silica gel, calcium carbonate, or dolomite.

FIG. 5 shows that CA obtained from blood obtained from steak immobilised to silica gel in sea water in acidic conditions is active.

FIG. 6 shows 6×50 mL falcon tubes containing either 0, 1, or 10 mL of crude CA from a commercial source added to 0.5 g of raw “R” or processed “P” basalt, supplemented with 0.1 M NaPi pH 7.0 (25 mL).

FIG. 7 shows a bar graph showing that CA obtained from animal blood immobilised on CaCO3 (chalk) can turn over 4-NPA product up to 60° C.

FIG. 8 shows a bar graph showing CA-containing samples produced more CO2 compared to the buffer only controls.

FIG. 9 shows a bar graph showing variation of measured % CO2 equivalents using different basalt carriers. Rb: Remin basalt; Db: Duntilland basalt; SG: silica gel.

FIG. 10 shows a bar graph showing variation of measured % CO2 equivalents when using different basalt carriers and steel slag with tap water or buffer during the CA reaction.

FIG. 11 shows a bar graph showing variation of measured % CO2 equivalents when using different basalt carriers, including basalt from Dentilland, Remin, and Crag Mill, with tap water during the CA reaction.

FIG. 12 shows a bar graph showing the effect of different process conditions on CO2 capture by CA immobilised on Dentilland basalt performed in packed bed reactor. “w.r.t” means “with respect to”.

FIG. 13 shows a bar graph showing the effect of CO2 gas flow on the CO2 capture by CA immobilised on Dentilland basalt performed in packed bed reactor. “w.r.t” means “with respect to”.

FIG. 14 shows a bar graph showing the enzyme activity of supernatant (Sup after . . . ), wash fractions (Wash 1 and Wash 2) and the supernatant after reaction (Sup after the reaction) were determined using 4NPA assay.

FIG. 15A shows a bar graph showing variation of 4NPA activity of samples taken from the plots of the field pre-trial after 1 week and 2 weeks post-application of the CA source to the soil. The numbers on the x-axis denote plot numbers, which are provided in Table 3.

FIG. 15B shows a bar graph showing variation of CO2 equivalents of samples taken from the plots of the field pre-trials after 1 week and 2 weeks post-application of the CA source to the soil. The numbers on the x-axis denote plot numbers, which are provided in Table 3.

FIG. 15C shows a bar graph showing percentage carbonate formation measured from samples taken from plots of the field pre-trials after 2, 4, and 6 weeks post-application of the CA source to the soil.

### DETAILED DESCRIPTION OF THE INVENTION

The disclosure is based on the development of new methods and compositions to sequester carbon in enhanced weathering by application of Carbonic Anhydrase, and optionally rock, to material such as soil or earth. This accelerates the rate of carbon sequestration.

In one aspect of the invention, Carbonic Anhydrase (and optionally rock) is applied to soil or earth. Advantageously, immobilisation of the Carbonic Anhydrase onto a solid support retains the Carbonic Anhydrase in the top soil. Similarly, engineering of the Carbonic Anhydrase to retain it at a defined soil depth is also advantageous.

Another aspect of the invention provides a mixture comprising Carbonic Anhydrase and seed, optionally wherein the Carbonic Anhydrase is immobilised to a solid support that may optionally be the seed. Another aspect of the invention provides a mixture comprising Carbonic Anhydrase and soil, optionally wherein the Carbonic Anhydrase is immobilised to a solid support.

Another aspect of the invention provides a mixture comprising Carbonic Anhydrase and rock, optionally wherein the Carbonic Anhydrase is immobilised to a solid support that may optionally be the rock.

Another aspect of the invention provides a mixture comprising carbonic anhydrase, rock and soil, optionally wherein the Carbonic Anhydrase is immobilised to a solid support.

Carbonic anhydrase enzymes are well-known in the art. This enzyme was first identified in 1933, in red blood cells of cows. Since then, it has been found to be abundant in all mammalian tissues, plants, algae and bacteria. This ancient enzyme has three distinct classes (called alpha, beta and gamma carbonic anhydrase). Members of these different classes share very little sequence or structural similarity, yet they all perform the same function and typically require a zinc ion at the active site.

PDB entries 1ca2, 1ddz and 1thj are examples of the alpha, beta and gamma Carbonic Anhydrase enzymes, respectively.

In some embodiments, the CA is from an animal species, for example cattle or sheep.

In some embodiments, the CA enzyme may be obtained, isolated or purified from animal blood, typically non-human mammalian blood, for example bovine or ovine blood.

In some embodiments, the CA enzyme may be produced recombinantly, typically expression of an exogenous gene in a host species. The host species is typically a microbial cell and/or the enzyme gene is expressed under a controllable promoter. For example, a mammalian carbonic anhydrase can be expressed in bacterial (e.g. E. coli) or yeast (e.g. S. cerevisiae or S. pombe) cell, typically on a recombinant expression cassette.

Some aspects of the invention involve one or more of the following numbered features:
1. Enzyme+solid support: identify pairing with retained enzyme activity and greatest imparted stability
2. Select solid support to improve longevity of the accelerated enzyme effect on the pre-biologically treated version of enhanced weathering (whatever rock and/or system is being deployed)
3. Select solid support to prevent leaching too deep into the soil, which is then too far from the enhanced weathering environment
4. Select solid support to prevent deactivation and/or degradation by protease or other active entities in the environment of the enhanced weathering process.

### Enzymes

The term enzyme is to be given its usual meaning in the art, i.e. a protein that accelerates or catalyses chemical reactions. An enzyme may have one or more active sites that bind to a substrate or selection of substrates. An enzyme may be naturally occurring or it may be of synthetic origin.

An enzyme is capable of catalysing a given reaction, and is sometimes thereof rereferred to as “an activity” or an “enzyme activity”. For example, a protein capable of catalysing the conversion of carbon dioxide and water into bicarbonate and protons is a Carbonic Anhydrase activity.

Suitable enzymes can include, for example: enzymes comprising natural and unnatural amino acids, chemical modifications or post-translational modifications; natural enzymes; wild-type enzymes; recombinant enzymes; enzymes produced by directed evolution, de novo design or the genetic fusion of peptide or protein domains; peptide catalysts; nucleic acid enzymes (e.g. ribozymes or DNA enzymes); hybrid catalysts; monomeric, dimeric or multimeric enzymes; enzymes produced in vivo or in vitro for example by solid-phase synthesis.

In an active enzyme system, the enzymes are provided under conditions suitable for enzyme activity, for example in the necessary physical and chemical conditions for enzyme activity. For the reaction to proceed, reagents will be needed, along with any necessary cofactor such as Zinc. Metal ions may also be included in the active enzyme system.

In the present invention, the enzyme is typically carbonic anhydrase.

In some embodiments, the Carbonic Anhydrase comprises an animal carbonic anhydrase. Optionally, the animal Carbonic Anhydrase comprises a mammalian carbonic anhydrase, optionally bovine erythrocyte Carbonic Anhydrase and/or human carbonic anhydrase.

In some embodiments, the Carbonic Anhydrase comprises a plant carbonic anhydrase. In some embodiments, the Carbonic Anhydrase comprises a bacterial carbonic anhydrase.

In some embodiments, the Carbonic Anhydrase comprises an alpha carbonic anhydrase. In some embodiments, the Carbonic Anhydrase comprises a beta carbonic anhydrase. In some embodiments, the Carbonic Anhydrase comprises a gamma carbonic anhydrase. In some embodiments, the Carbonic Anhydrase comprises a delta carbonic anhydrase. In some embodiments, the Carbonic Anhydrase comprises a zeta carbonic anhydrase.

In some embodiments, the Carbonic Anhydrase used in the method of the present invention comprises one or more classes of carbonic anhydrase.

### Enzyme Activity and Stability

An enzyme in an active state, i.e. an active enzyme, is capable of catalysing a chemical reaction.

In one embodiment, an enzyme is stable if it remains in an active state for a period of time. In some embodiments, the period of time is at least a day, at least three days, at least 1 week, at least 2 weeks, at least 1 month, at least 6 weeks, at least 2 months, at least 3 months, at least 6 months, at least 9 months, at least 12 months, at least 18 months or at least 2 years, optionally wherein the period of time is at least one month or at least one year.

In some embodiments, the enzyme is active when present in soil as described herein, typically exposed to the elements and weather.

In some embodiments, the activity of an enzyme can be measured by the yield of the product produced by the enzymatic reaction, or by measuring conversion, depletion or consumption of the substrate. In some embodiments, the enzyme is active if the yield of product is about 40% or more, about 50% or more, about 60% or more, about 70% or more, about 80% or more, about 90% or more, or about 100% of the yield from the initial use cycle, when assessed under the same reaction conditions.

In some embodiments, the enzyme is stable over a given time period if, over that time period, the yield of product is about 40% or more, about 50% or more, about 60% or more, about 70% or more, about 80% or more, about 90% or more, or about 100% of the activity within one hour of immobilisation, when assessed under the same reaction conditions.

In some embodiments, the enzyme is active after 4 weeks or three months. In some embodiments, the enzyme is active over that time period if the yield of product is about 40% or more, about 50% or more, about 60% or more, about 70% or more, about 80% or more, about 90% or more, or about 100% of the activity within one hour of immobilisation, when assessed under the same reaction conditions.

In one aspect of the invention, a homologue of a reference enzyme has higher stability than the reference enzyme under the specified reaction process conditions. For example, a reference enzyme from a first species may catalyse a stated reaction when immobilised, but a homologue of that enzyme catalyses the same stated reaction with higher activity when immobilised to the same solid support. Typically, the homologue enzyme will have higher activity after a period of time, for example after weeks or months, and the long-term stable activity can be more valuable in terms of the efficiency of large scale biocatalysis than the initial activity shortly after immobilisation. Therefore, identifying homologues that are active and stable for a period of weeks, months or longer, is particularly advantageous.

### Identification of One or More Homologues

In one embodiment, an enzyme for immobilisation is identified by identifying two or more enzymes with different polypeptide sequences that catalyse a chemical reaction of interest. In some embodiments, the two or more enzymes comprises the enzyme of interest and one or more enzymes that are homologues of that enzyme of interest. In some embodiments, identifying the two or more enzymes is performed in a search, typically a search of one or more bioinformatic databases using one or mor bioinformatic search tools.

Bioinformatic databases and tools are well-known in the art. For example, a summary of useful bioinformatics tools can be found at www.ebi.ac.uk/tools and with sequence similarity searching tools collected at https://www.ebi.ac.uk/Tools/sss/

In some embodiments, identifying the two or more enzymes is performed in a search wherein an amino acid sequence is an input. In some embodiments, identifying two or more enzymes is performed in a search wherein a nucleic acid sequence is an input. The input sequence is typically a reference enzyme sequence from a first species. In some embodiments, the search output is one or more amino acid sequences. In some embodiments, the search output is one or more nucleic acid sequences. Typically, the search output includes an enzyme sequence from a different species from the reference sequence, that has a different sequence from the sequence of the reference enzyme, and that catalyses the same chemical reaction.

In some embodiments, the search comprises use of one or more sequence similarity searching methods. Optionally, the sequence similarity searching method is one or more of: BLAST (Basic Local Alignment Search Tool), optionally NCBI BLAST, PSI-BLAST, or PHI-BLAST; FASTA, optionally FASTA, SSEARCH, PSI-SEARCH, PSI-SEARCH2, GGSEARCH, GLSEARCH, or FASTM/S/F.

In some embodiments, the search comprises a classical literature search to identify additional mutations of the enzyme of interest.

In some embodiments, identifying the two or more enzymes comprises search which comprises one or more, optionally all, of use of one or more sequence similarity searching methods, alignment of multiple sequences, identifying an enzyme of interest and identifying sequences that are close to the enzyme of interest using a database of protein families, and identify additional mutations of the enzyme of interest. Optionally, two or more sequences are aligned again to remove redundancies.

### Selection of One or More Homologues

The present invention may involve the identification and selection of an enzyme and a solid support to which the enzyme can be immobilised.

In one aspect of the invention, the enzyme is identified by a method that comprises a step comprising identifying two or more enzymes with different polypeptide sequences that catalyse a chemical reaction of interest. In some embodiments, the two or more enzymes comprises one enzyme which is the enzyme of interest and one or more enzymes which are homologues of the enzyme of interest.

In some embodiments, properties of the one or more homologues are compared to properties of the enzyme of interest to select for one or more homologues for immobilisation.

Enzymatic activity is known to be influenced by factors including, but not limited to, temperature. Typically, the optimal temperature for an enzyme is the temperature which allows the enzyme to function at maximal activity.

In some embodiments, the enzyme is derived from a mesophilic organism. In some embodiments, the enzyme is derived from a cryophilic organism.

In some embodiments, the enzyme or homologue is mesophilic. In some embodiments, the enzyme or homologue is cryophilic.

In some embodiments, the optimum temperature of the enzyme or homologue is 70° C. or less, for example between 40° C. and 70° C. . . . In some embodiments, the optimum temperature of the enzyme or homologue is 50° C. or less, for example between 40° C. and 50° C. In some embodiments, the optimum temperature of the enzyme or homologue is 40° C. or less, for example between 30° C. and 40° C. In some embodiments, the optimum temperature of the enzyme or homologue is 30° C. or less, for example between 20° C. and 30° C. In some embodiments, the optimum temperature of the enzyme or homologue is 20° C. or less, for example between 10° C. and 20° C. In some embodiments, the optimum temperature of the enzyme or homologue is 15° C. or less, for example between 5° C. and 15° C. In some embodiments, the optimum temperature of the enzyme or homologue is 5° C. or less, for example between minus 20° C. and plus 5° C. or between minus 20° C. and 0° C.

In a further embodiment, such enzymes or homologues are engineered to improve their stability.

### Selection of Enzyme or Homologue According to Temperature of Geographical Region

Enzymatic activity is known to be influenced by factors including, but not limited to, temperature. Typically, the optimal temperature for an enzyme is the temperature which allows the enzyme to function at maximal activity.

The present invention may involve the identification and selection of an enzyme according to the temperature of the geographical location or region to which the enzyme is to be applied. The selection of the enzyme may be determined according to one or more of previous temperature recordings, current temperature recordings, and predictions of future temperature. In some embodiments, the temperature comprises or consists of the temperature of air. In some embodiments, the temperature comprises or consists of the temperature of top soil. In some embodiments, the temperature comprises or consists of the average temperature of the geographical location or region. In some embodiments, the average temperature comprises or consists of a yearly average. In some embodiments, the average temperature comprises or consists of the average temperature over the course of a growing season of a crop. In some embodiments, the average temperature comprises or consists of the day average temperature. In some embodiments, the average temperature comprises or consists of the night average temperature. In some embodiments, the average temperature comprises or consists of the day average temperature and the night average temperature.

Typically, methods of sequestering carbon according to the present invention may occur in geographical locations or regions where the average temperature is about 70° C. or less. Accordingly, an enzyme or homologue that has an optimal activity at a temperature of about 70° C. or less may be selected. In some embodiments, the optimal activity of the enzyme or homologue occurs at 70° C. or less. In some embodiments, the optimal activity of the enzyme or homologue occurs at 60° C. or less. In some embodiments, the optimal activity of the enzyme or homologue occurs at 50° C. or less. In some embodiments, the optimal activity of the enzyme or homologue occurs at 40° C. or less. In some embodiments, the optimal activity of the enzyme or homologue occurs at 30° C. or less. In some embodiments, the optimal activity of the enzyme or homologue occurs at 20° C. or less. In some embodiments, the optimal activity of the enzyme or homologue occurs at 15° C. or less. In some embodiments, the optimal activity of the enzyme or homologue occurs at 10° C. or less. In some embodiments, the optimal activity of the enzyme or homologue occurs at 5° C. or less.

Different homologues may be selected for use in a method of sequestering carbon according to the present invention, depending on the geographical location. For example, a homologue with a lower optimal temperature may be preferentially selected for use in a colder geographical location than a warmer geographical location.

Accordingly, in some embodiments, the optimal temperature for the enzymatic activity of an enzyme or homologue corresponds to the average temperature of the geographical location or region to which the enzyme is applied or will be applied. In some embodiments, the optimal temperature is within 20° C. of the average temperature of the geographical location or region. For example, if the average temperature of the geographical location or region is 20° C., the optimal temperature for enzyme activity would fall between 0° C. and 40° C. In some embodiments, the optimal temperature is within 15° C. of the average temperature of the geographical location or region. In some embodiments, the optimal temperature is within 10° C. of the average temperature of the geographical location or region. In some embodiments, the optimal temperature is within 5° C. of the average temperature of the geographical location or region.

### Cells Comprising Carbonic Anhydrase

The disclosure typically contemplates the use of an isolated, purified or partly-purified carbonic anhydrase enzyme, for example that has been extracted from its natural environment by an isolation or purification process. This may be purification such that the carbonic anhydrase is the majority component, or substantially the only component, of a preparation following a purification step (e.g. the enzyme has been purified using one or more chromatography steps as are well-known in the art). The enzyme may alternatively have been only slightly isolated from the cell in which it was expressed, for example the enzyme can be provided in a lysate.

In some embodiments the enzyme is not separated from the cell in which it is expressed. The carbonic anhydrase is applied to the soil while still inside a cell, i.e. a cell comprising the enzyme is applied to the soil. The cell may be alive or dead. The cell should contain carbonic anhydrase protein, and/or have the function of being able to express carbonic anhydrase under appropriate conditions. This ability requires the presence of a nucleic acid sequence that encodes a carbonic anhydrase enzyme. The carbonic anhydrase may be endogenous to the cell, or may be exogenous (e.g. expressed recombinantly).

Typically, the cell is a microorganism. In this aspect of the invention, a method of sequestering atmospheric carbon comprises the step of applying a microbe and optionally rock to soil, wherein the microbe expresses (or has previously expressed, or is able to express) carbonic anhydrase. Typically, the microbe endogenously expresses carbonic anhydrase.

The microbe typically comprises one or more single-celled organisms, optionally a prokaryotic cell, a fungal cell, a microalgae cell, an archaea cell, and/or a bacterial cell.

In some embodiments, the microbe:
is obtained from or is present in milk, fermented milk, yogurt or kefir;
comprises or consists of lactobacillus, lactococcus, leuconostoc, streptococcus or enterococcus bacteria;
comprises or consists of Saccharmocyes cerevisiae, baker's yeast, brewer's yeast or Schizosaccharomyces pombe;
comprises or consists of compost starter containing organisms;
comprises or consists of a combination of at least one bacterium and at least one fungus, optionally wherein the at least one bacterium comprises an Actinomycetes.

Table 3 in the Examples below demonstrates the use of carbonic-anhydrase expressing cells from a wide range of sources, including chicken serum, potatoes, bakers' yeast, brewers' yeast, compost starter, yogurt (comprising Lacticaseibacillus casei Shirota), eggs, raw goats milk, beef, pork, raw cows milk and grass. Each of these sources of CA is provided as a separate embodiment of the invention. These cells themselves can be used according to the present disclosure, or the enzyme can be extracted, isolated or purified and used as a purified enzyme preparation or a part-purified extract.

In some embodiments, the cell comprising carbonic anhydrase may be immobilised to rock, typically particulate rock. Any technique of immobilising the cell to the rock can be used, for example adsorption.

### Immobilised Enzymes

Compared to free enzymes in solution, immobilised enzymes are typically more robust and more resistant to environmental changes.

Multiple copies of a single enzyme may be attached to a single support, for example a single bead or other solid support as described herein e.g. waste material such as a corn husk or fragment thereof, the rock component itself, a seed, or a plant. These may be attached in an identical fashion, or different copies of a single functional molecule may be attached differently. This can provide, for example, improved activity for a single type of functional molecule when the structure is placed under different bulk environmental conditions.

In some embodiments, two or more different Carbonic Anhydrase enzymes (i.e. different sequences) are attached to each support, e.g. each bead, each rock particle, each seed, or each plant. These may each be present once, or may each be present multiple times.

### Solid Supports

The invention typically involves immobilising an enzyme, typically Carbonic Anhydrase, to a solid support. The solid support may comprise controlled pore glass, polystyrene, sepharose or alginate. In some embodiments, the solid support is particulate. The solid support may comprise beads, for example, controlled pore glass beads, polystyrene beads or buoyant beads. In some embodiments, the solid support is a resin. In some embodiments, the solid support is magnetic, for example magnetic beads. In one embodiment, the solid support comprises Poly(styrene-divinylbenzene).

Typically, the solid support is available at a low cost to ensure commercial viability of the method.

In some embodiments, the enzyme is directly immobilised onto the rock, e.g. finely ground rock, used for the enhanced weathering process.

### Plants and Seeds as Solid Supports

Without wishing to be bound by theory, the inventors have found that inoculating an enzyme such as Carbonic Anhydrase onto a plant seed or plant root has many advantages over microbial inoculations, including one or more of:
the enzyme is easier to scale than the microbial inoculant in an expression vessel.
the enzyme dosing onto the seed or plant can be controlled much more easily than inoculated microbial systems in the soil
the enzyme might be more efficient than other related microbial approaches at helping to sequester carbon for agricultural fields
the enzyme can be modified for improved properties and still considered non-GMO compared to the microbial inoculant.

The inventors also consider, without being bound by theory, that the enzyme might be more active deeper in the soil near the plant roots where biomass decomposition leads to enhanced CO2 levels.

Notably, rock such as basalt is not required for Carbonic Anhydrase enzyme to be inoculated on seeds or plant. The salinity+CO2+water in the soil is suitable to carry out the carbon sequestration. Rock may be added in some embodiments.

In certain embodiments, the plant or seed is sprayed with a liquid comprising the enzyme, wherein the enzyme is typically carbonic anhydrase. Methods of spraying plants and plant seeds are known in the art.

### Seed as a Solid Support

In one aspect the solid support is a seed. In some embodiments, the enzyme is immobilised onto a seed. Typically, the seed is a variety used in agriculture. In some embodiments, the enzyme is directly immobilised onto the seed. In some embodiments, the enzyme is directly immobilised onto the seed coat.

Certain non-limiting aspects and embodiments relate to coating, dipping or spraying agricultural seeds with a liquid containing Carbonic Anhydrase enzyme, which then achieves immobilization of the enzyme onto the hard shell of the seed before it is placed in the soil. The Carbonic Anhydrase enzyme then remains in/around the plant root system for a prolonged period of time, for example one month or more.

Typically, the enzyme is immobilised onto the seed before the seed is sown. Accordingly, in some embodiments, the seed is contacted with a mixture comprising the enzyme, typically a liquid mixture. In some embodiments, the seed is sprayed with the mixture. In some embodiments, the seed is stirred with or into the mixture. In some embodiments, the seed is introduced to the mixture and the mixture is aerated. In some embodiments, the seed is submerged in the mixture. In some embodiments, the seed is placed on an article comprising the mixture. In some embodiments, the seed is placed within an article comprising the mixture. In some embodiments, the seed is placed underneath an article comprising the mixture. In some embodiments, the seed is placed on an article soaked in the mixture. In some embodiments, the seed is placed within an article soaked in the mixture. In some embodiments, the seed is placed underneath an article soaked in the mixture. Following contact with a liquid comprising the enzyme, the seed is typically removed from contact with the enzyme liquid, and may optionally be allowed to dry in ambient surroundings or be subjected to an active drying step.

Some agricultural crop seeds undergo seed priming prior to sowing. Seed priming is a process of regulating the germination process by managing the temperature and seed moisture content. In some embodiments, the seed is contacted with the enzyme (e.g. mixture comprising the enzyme) prior to priming. In some embodiments, the seed is contacted with the mixture during priming. In some embodiments, the seed is contacted with the mixture after priming.

Accordingly, the present disclosure provides a mixture comprising Carbonic Anhydrase and seed, wherein the Carbonic Anhydrase is immobilised to the seed. In some embodiments, the mixture is sown into agricultural land.

Immobilising the enzyme to a seed coat may be particularly useful commercially, as this may allow for reduced labour time on agricultural land and/or reduce the need for additional steps to supply the enzyme to the top soil separately to the seed.

### Plant as a Solid Support

In another aspect, the solid support is a plant. Typically, the plant is suitable for growing as an agricultural crop. In some embodiments, the enzyme is directly immobilised onto the plant. The enzyme may be immobilised onto one or more parts of the plant, including but not limited to, the plant root, the plant stem, the plant leaf, the plant bulb, and the plant tuber. In some embodiments, the enzyme is directly immobilised onto the plant stem. In some embodiments, the enzyme is directly immobilised onto the plant bulb. In a further embodiment, the enzyme is directly immobilised onto the bulb tunic. In some embodiments, the enzyme is directly immobilised onto the plant tuber. In some embodiments, the enzyme is directly immobilised onto the plant tuberous root. In a further embodiment, the enzyme is directly immobilised onto the tuber periderm. In a preferable embodiment, the enzyme is directly immobilised onto the plant root. In some embodiments, the plant is a seedling. In some embodiments, the plant is a mature plant.

The enzyme may be immobilised onto the plant before it is planted in agricultural land. Accordingly, in some embodiments, the plant is contacted with a mixture comprising the enzyme. In some embodiments, the plant is sprayed with the mixture. In some embodiments, the plant is dipped into the mixture. In some embodiments, the plant is submerged in the mixture. In some embodiments, the plant is watered with the mixture. For example, the enzyme may be immobilised onto the plant root by contacting the plant root with a mixture comprising the enzyme, optionally wherein the contacting occurs by one or more of spraying, dipping, submerging, and watering.

The enzyme may be immobilised onto the plant after it is planted in agricultural land. Accordingly, in some embodiments, the plant is contacted with a mixture comprising the enzyme. In some embodiments, the plant is sprayed with the mixture. In some embodiments, the plant is watered with the mixture. In some embodiments, the mixture is provided to the plant through an irrigation system. For example, the enzyme may be immobilised onto the plant root by contacting the plant root with a mixture comprising the enzyme, optionally wherein the contacting occurs by watering the plant with the mixture, optionally wherein the watering is performed using an irrigation system.

### Species of Plants and Seeds

In some embodiments, the plant or seed is a crop plant or crop plant seed, optionally a crop plant of agronomic importance which is cultivated for food, animal feed, fiber, fuel, and/or industrial purposes. In one embodiment, the crop plant is a commercial crop plant. A commercial crop plant is a plant cultivated to produce a harvested horticultural product that is for sale and/or profit, as well as subsistence crops which may be grown to support other agricultural products, such as livestock.

In one embodiment, the plant is a commercial crop plant. In one embodiment, the commercial crop plant is a compatible crop selected from the group consisting of species of the genus Triticum, Brassica, Gossypium, Zea, Corchorus, Saccharum, Medicago, Lolium, Coffea, Camellia, Oryza, Hordeum, Boehmeria, Nicotiana and Cannabis.

In one embodiment, the commercial crop is selected from the group consisting of the species Triticum aestivum, Brassica napus, Brassica rapa, Brassica juncea, Gossypium hirsutum, Gossypium barbadense, Gossypium arboretum, Gossypium Herbaceum, Zea mays, Medicago sativa, Lolium multiflorum, Corchorus capsularis, Saccharum officinarum, Cannabis sativa, Coffea Arabica, Coffea Robusta, Camellia sinensis, Oryza sativa, Hordeum vulgare, Boehmeria nivea and Nicotiana tabacum.

In one embodiment, the crop plant is a cereal plant. Cereal plants include, for example, wheat [Triticum), rice [Oryza), barley [Hordeum), corn [Zea).

In one embodiment, the cereal plant is selected from wheat, and barley.

In one embodiment, the cereal plant is a wheat plant.

The inventors have found that growing a plant or seed that has been contacted with an enzyme such as Carbonic Anhydrase results in an increase in soil carbon and/or an increase in yield of the plant being cultivated in the soil. The plants may be crop plants, which are known in the art and include for example wheat, canola, and barley. Applying the enzyme to the plant or seed can result in increased yield of plant relative to untreated plants. The inventors have further found that the soil in which these plants are grown can have increased organic carbon content relative to soil in which untreated plants are grown.

In some embodiments, the present disclosure to enhancement of agricultural soils, mitigating atmospheric carbon dioxide, and providing agronomic benefits to crop plants. Additionally, the present disclosure relates in part to methods and compositions for increasing soil organic carbon in soil, and increasing crop plant yield.

In some embodiments, the soil is contacted with the enzyme. The soil may be contacted with the enzyme prior to planting a plant, for example before, during, or after tilling the soil in preparation for planting. In other embodiments, the soil may be contacted with the enzyme after the plant has been planted. In some embodiments, the soil is contacted with the enzyme by planting in the soil plants that have been contacted with the enzyme.

In some embodiments, the step of contacting with the enzyme comprises applying the enzyme to seeds of the plant prior to planting.

In some embodiments, the step of contacting with the enzyme comprises applying the enzyme to seedlings of the plant.

In some embodiments, the step of contacting with the enzyme comprises applying the enzyme to a plot of soil that is cultivated, such that the enzyme is retained by the soil as the crops are rotated, even in the absence of crops for periods of time.

In one embodiment, the plants are contacted with the enzyme as a seed coating before, during or after one or more of the stages of germination of a seed, or as a root inoculant of a seedling. For example, the enzyme may be applied as a seed coating to seeds at large scale prior to sowing a crop.

In some embodiments, the enzyme is provided as a composition comprising an agriculturally acceptable excipient.

As used herein, an “agriculturally acceptable excipient” refers to an essentially inert substance that can be used as a diluent and/or carrier for the enzyme and is not detrimental to the plant or seed.

In one embodiment, the agriculturally acceptable excipient is a solid or liquid carrier. Suitable solid carriers include mineral earths (e.g., calcium phosphate, calk, clay, diatomaceous earth, dolomite, kaolin, silicates, silica gels, talc, etc.), cellulose, and starch. Suitable liquid carriers include water, or any other liquid solvents which are not toxic to the seed or the plant.

In one embodiment, the composition comprises an agriculturally acceptable additive. As used herein, an “agriculturally acceptable additive” is an additive that may assist in or enhance the performance of the enzyme and/or the plant or seed, and is not detrimental to the one or more enzyme or to the plant or seed. Examples of agriculturally acceptable additives include preservatives, stabilizers, nutrition enhancers, wetter-spreaders, stickers (adhesives), penetrants, root promoters, fungicides, urea, fertilisers, pesticides, fulvic acid, humus, nanoparticles/nanomaterials.

In some embodiments, the enzyme may be provided as a composition in the form of a dried powder, a spray, a slurry, a sachet, a liquid, a jelly, a seed coating, an enhancer, and/or combinations thereof. In some embodiments, the composition is in the form of a seed coating, a foliar spray, granule, powder, soil drench or a root dip.

In some embodiments, the composition is in the form of a seed coating.

In some embodiments, the composition is in the form of a foliar spray.

In some embodiments, the composition is in the form of a soil drench.

In some embodiments, the composition is in the form of a root dip.

### Solid Support Cost

The invention typically utilises solid supports that are available at a low cost to ensure commercial viability of the method when applied at a large scale, for example to be spread over acres or hectares of ground.

In some embodiments, the solid support has a procurement cost of $20 or less per kg. Typically, the solid support has a procurement cost of $10 or less per kg. It is beneficial if the solid support has a procurement cost of $5 or less per kg and beneficial further still if the solid support has a procurement cost of $1 or less per kg, or $0.5 or less per kg.

### Source of Alternative Solid Supports

In some embodiments, the solid support comprises plant matter. In some embodiments, the plant matter comprises vegetable matter, optionally fibrous matter. In some embodiments the vegetable matter comprises waste matter from the farming process and/or food chain. In some embodiments, the waste matter comprises corn husks.

In some embodiments, the solid support comprises animal matter, optionally fibrous matter.

Typically, the solid support is obtained from waste matter. In some embodiments, the waste matter comprises or consists of produce such as fruit and/or vegetables (e.g. corn or corn husks) that are too old, spoiled or rotten to be sold, or are inedible. In some embodiments, the waste matter comprises fibrous plant or animal matter that is too old, spoiled or rotten to be sold, or is inedible. In certain embodiments, the solid support is from material that would otherwise be seen as low or no value, or as a waste product. In some embodiments, the solid support is from commercial waste or household waste.

Typically, the material is robust enough not to degrade significantly, or at all, over a period of at least 6 months, or least one year.

### Bead Solid Supports

In some embodiments, the solid support is a bead. Solid supports that are beads are well-known in the art. In some embodiments, the solid support is a resin bead. Resin beads are well-known in the art. Resin beads are typically essentially spherical.

In some embodiments, the bead is porous. In porous beads, binding sites can also be internal, leading to higher loading of enzyme.

The beads may be from 10 micron to 1500 micron diameter, typically 100 micron to 1200 micron, for example 150 micron to 300 micron diameter.

In some embodiments, the beads are methacrylate beads, for example epoxy methacrylate beads, epoxy/butyl methacrylate beads, amino C2 methacrylate beads or Amino C6 methacrylate beads.

The inventors have successfully immobilised enzymes using a variety of beads including amine-functionalised beads and epoxy-functionalised beads.

### Epoxy Resins

Beads that are functionalised with epoxides are known as “epoxy” functionalised resins, or epoxy-resins. The IUPAC name for an epoxide group is an oxirane. Epoxides react with nucleophiles and in the context of protein immobilisation, the reactive residues will typically be Lys and Cys (and sometimes His). Surface epoxy groups on beads therefore allow direct covalent binding of proteins or peptides, which can be achieved by simple incubation of the protein or peptide with the epoxy-bead, for example overnight.

Epoxy-functionalised beads can be used in the invention because they form very stable covalent linkages.

In some embodiments, the bead is an epoxy-methacrylate bead. In some embodiments, the bead is an epoxy/butyl methacrylate bead.

In some embodiments, the pore diameter of the epoxy beads is 300 to 1800 Å. Optionally, the pore diameter is 300 to 600 Å, 450 to 600 Å, or 1200 to 1800 Å. In some embodiments, the total moisture of the beads is 50 to 80%.

Optionally, the total moisture of the beads is 50 to 65%, or 70 to 80%.

In some embodiments, a minimum of 85% of the beads are perfectly spherical. Optionally a minimum of 95% of the beads are perfectly spherical.

In one embodiment, the epoxy beads are epoxy/butyl methacrylate beads, wherein the particle size range is 250 to 1000 micron, the pore diameter is 450 to 650 Å, and the total moisture is 70 to 80%. The functional group is epoxy. The spherical beads are stable at pH 5 to pH 9, and are suitable for storage at 2 to 8° C. Optionally, a minimum of 85% of the beads are perfectly spherical.

In one embodiment, the epoxy beads are epoxy methacrylate beads, wherein the particle size range is 150 to 300 micron, the pore diameter is 300 to 600 Å, and the total moisture is 50-65%. The functional group is epoxy. The spherical beads are stable at pH 5 to pH 9, and are suitable for storage from 2 to 8° C. Optionally, a minimum of 95% of the beads are perfectly spherical.

In one embodiment, the epoxy beads are epoxy methacrylate, wherein the particle size range is 150 to 300 micron, the pore diameter is 1200 to 1800 Å, and the total moisture is 70 to 80%. The functional group is epoxy. The spherical beads are stable at pH 5 to pH 9, and are suitable for storage at 2 to 8° C. Optionally, a minimum of 95% of the beads are perfectly spherical.

### Amine-Functionalised Resins

Beads that are functionalised with amines are known as amine-functionalised beads or amine-functionalised resins. Amine functional groups form covalent amide bonds with proteins or polypeptides. Amine-functionalised beads are generally pre-activated with glutaraldehyde, prior to ionic polypeptide immobilisation. pH adjustment during incubation results in the formation of an amide bond between the bead and the protein or polypeptide.

In some embodiments, the bead is an amino C2 methacrylate bead. In some embodiments, the bead is an amino C6 methacrylate bead.

In some embodiments, the particle size range of the amine-functionalised beads is 150 to 300 micron.

In some embodiments, the pore diameter is 600 to 1800 Å. Optionally, the pore diameter is 600 to 1200 Å, or 1200 to 1800 Å.

In some embodiments, the total moisture of the beads is 62 to 80%. Optionally, the total moisture is 62 to 72%, or 70 to 80%.

In some embodiments, a minimum of 95% of the beads are perfectly spherical.

In one embodiment, the amine-functionalised beads are amino C2 methacrylate beads, wherein the particle size range is 150 to 300 micron, the pore diameter is 600 to 1200 Å, and the total moisture is 62 to 72%. The functional group is amino (short spacer). The beads are typically stable at pH 3 to pH 10, and suitable for storage at 2 to 20° C. Optionally, a minimum of 95% of the beads are perfectly spherical.

In one embodiment, the amine-functionalised beads are amino C2 methacrylate beads, wherein the particle size range is 150 to 300 micron, the pore diameter is 1200 to 1800 Å, and the total moisture is 70 to 80%. The functional group is amino (short spacer). The beads are typically stable at pH 3 to pH 10, and are suitable for storage at 2 to 20° C. Optionally, a minimum of 95% of the beads are perfectly spherical.

In one embodiment, the amine-functionalised beads are amino C6 methacrylate beads, wherein the particle size range is 150 to 300 micron, the pore diameter is 1200 to 1800 Å, and the total moisture is 70 to 80%. The functional group is amino (short spacer). The spherical beads are stable at pH 3 to pH 10, and are suitable for storage at 2 to 20° C. Optionally, a minimum of 95% of the beads are perfectly spherical.

### Adsorbent Resins

In some embodiments, the bead is an adsorbent resin bead. Adsorbent beads immobilise the protein or peptide by adsorption. Proteins or polypeptides with hydrophobic properties can be efficiently immobilised onto adsorbent beads. Adsorbent resins are water-insoluble carriers that allow physical adsorption of an enzyme onto its surface.

In some embodiments, the bead is a polystyrenic, non-functionalised resin bead. In some embodiments, the bead is an octadecyl methacrylate bead. In some embodiments, the bead is a polymethacrylic/divinylbenzene bead. In some embodiments, the bead is a polymethacrylic/divinylbenzene macroporous bead. In some embodiments, the polymer structure is polymethacrylic crosslinked with divinylbenzene.

Adsorbent beads can be used with the invention because they immobilise proteins or polypeptides with high mechanical stability.

In some embodiments, the particle size range is 300 to 1200 micron. Optionally, the particle size range is 300 to 710 micron, or 350 to 1200 micron.

In some embodiments, the pore diameter is 220 to 1200 Å. Optionally, the pore diameter is 900 to 1100 Å, 400 to 650 Å, 220 to 340 Å, or 350 to 1200 Å.

In some embodiments, the total moisture is 57 to 78%. Optionally, the total moisture is 67 to 78%, 58 to 63%, 57 to 68%, or 60-66%.

In some embodiments, the typical pore diameter by nitrogen adsorption is 300 Å. In some embodiments, the typical pore volume by nitrogen adsorption is 1.2 mL/g. In some embodiments, the typical surface area by nitrogen adsorption is 490 m2/g.

In some embodiments, the specific gravity is 1.1.

In some embodiments, a minimum of 95% of the beads are perfectly spherical.

In some embodiments, the adsorbent bead has a functional group. Optionally the adsorbent bead has an amino functional group. Optionally, the functional group is non-ionic.

In one embodiment, the adsorbent bead is a polystyrenic, non-functionalised resin bead, wherein the particle size range is 300 to 710 micron, the pore diameter is 900 to 1100 Å, and the total moisture is 67 to 78%. The spherical beads are stable at pH 1 to pH 14, and are suitable for storage at 2 to 20° C. Optionally, a minimum of 95% of the beads are perfectly spherical.

In one embodiment, the adsorbent bead is an octadecyl methacrylate bead, wherein the particle size range is 300 to 710 micron, the pore diameter is 400 to 650 Å, and the total moisture is 58 to 63%. The functional group is amino (short spacer). The spherical beads are stable at pH 2 to pH 10, and are suitable for storage at 2 to 20° C. Optionally, a minimum of 95% of the beads are perfectly spherical.

In one embodiment, the adsorbent bead is a polymethacrylic/divinylbenzene, non-functionalised resin bead, wherein the particle size range is 300 to 710 micron, the pore diameter is 220 to 340 Å, and the total moisture is 57 to 68%. The beads are typically stable at pH 1 to pH 14, and are suitable for storage at 2 to 20° C. Optionally, a minimum of 95% of the beads are perfectly spherical.

In one embodiment, the adsorbent bead is a polymethacrylic/divinylbenzene macroporous, adsorbent resin bead in a non-ionic form, wherein the particle size range is 350 to 1200 micron, the typical pore diameter by nitrogen adsorption is 300 Å, the typical pore volume by nitrogen adsorption is 1.2 mL/g, the typical surface area by nitrogen adsorption is 490 m2/g, the specific gravity is 1.1, and the moisture retention is 60-66%. The functional group is non-ionic and the polymer structure is polymethacrylic crosslinked with divinylbenzene. The spherical beads are stable at pH 0 to pH 14, and the temperature limit is 100° C.

### Ion Exchange Resins

In some embodiments, the bead is an ion-exchange resin. In some embodiments, the ion-exchange resin is a cation exchange resin or an anion exchange resin.

In some embodiments, the ion-exchange resin comprises polystyrene crosslinked with divinylbenzene.

Examples of epoxy, amine and adsorbent beads that are commercially available from Purolite Ltd. (Llantrisant, Wales, UK) are provided below along with their catalogue numbers.

### Epoxy Resins:

### ECR8204F

Epoxy-functionalised resin
Purolite “perfect bead”: min. 95%.
Epoxy methacrylate beads, wherein the particle size range is 150 to 300 μm, the pore diameter is 300 to 600 Å, and the total moisture is 50-65%. The functional group is epoxy. The spherical beads are stable at pH 5 to pH 9, and are suitable for storage from 2 to 8° C.

### ECR8285

Epoxy-functionalised resin
Purolite “perfect bead”: min. 85%.
Epoxy/butyl methacrylate beads, wherein the particle size range is 250 to 1000 μm, the pore diameter is 450 to 650 Å, and the total moisture is 70 to 80%. The functional group is epoxy. The spherical beads are stable at pH 5 to pH 9, and are suitable for storage at 2 to 8° C.

### ECR8215F

Epoxy-functionalised resin
Purolite “perfect bead”: min. 95%.
Epoxy methacrylate beads, wherein the particle size range is 150 to 300 μm, the pore diameter is 1200 to 1800 Å, and the total moisture is 70 to 80%. The functional group is epoxy. The spherical beads are stable at pH 5 to pH 9, and are suitable for storage at 2 to 8° C.

### Adsorbent Resins:

### ECR1090M

Adsorbent resin
Purolite “perfect bead”: min. 95%.
Functional group: none.
Polystyrenic, non-functionalised resin beads, wherein the particle size range is 300 to 710 μm, the pore diameter is 900 to 1100 Å, and the total moisture is 67 to 78%. The spherical beads are stable at pH 1 to pH 14, and are suitable for storage at 2 to 20° C.

### ECR8806M

Adsorbent resin.
Purolite “perfect bead”: min. 95%.
Octadecyl methacrylate beads, wherein the particle size range is 300 to 710 μm, the pore diameter is 400 to 650 Å, and the total moisture is 58 to 63%. The functional group is amino (short spacer). The spherical beads are stable at pH 2 to pH 10, and are suitable for storage at 2 to 20° C.

### ECR1030M

Adsorbent resin
Purolite “perfect bead”: min. 95%.
Functional group: none.
Polymethacrylic/divinylbenzene, non-functionalised resin beads, wherein the particle size range is 300 to 710 μm, the pore diameter is 220 to 340 Å, and the total moisture is 57 to 68%. The spherical beads are stable at pH 1 to pH 14, and are suitable for storage at 2 to 20° C.

### PAD610

Adsorbent resin
Polymethacrylic/divinylbenzene macroporous, adsorbent resin beads in a non-ionic form, wherein the particle size range is 350 to 1200 μm, the typical pore diameter by nitrogen adsorption is 300 Å, the typical pore volume by nitrogen adsorption is 1.2 mL/g, the typical surface area by nitrogen adsorption is 490 m2/g, the specific gravity is 1.1, and the moisture retention is 60-66%. The functional group is non-ionic and the polymer structure is polymethacrylic crosslinked with divinylbenzene. The spherical beads are stable at pH 0 to pH 14, and the temperature limit is 100° C.

### Amine Resins:

### ECR8309F

Amine-functionalised resin
Purolite “perfect bead”: min. 95%.
Amino C2 methacrylate beads, wherein the particle size range is 150 to 300 μm, the pore diameter is 600 to 1200 Å, and the total moisture is 62 to 72%. The functional group is amino (short spacer). The spherical beads are stable at pH 3 to pH 10, and suitable for storage at 2 to 20° C.

### ECR8315F

Amine-functionalised resin
Purolite “perfect bead”: min. 95%.
Amino C2 methacrylate beads, wherein the particle size range is 150 to 300 μm, the pore diameter is 1200 to 1800 Å, and the total moisture is 70 to 80%. The functional group is amino (short spacer). The spherical beads are stable at pH 3 to pH 10, and are suitable for storage at 2 to 20° C.

### ECR8415F

Amine-functionalised resin
Purolite “perfect bead”: min. 95%.
Amino C6 methacrylate beads, wherein the particle size range is 150 to 300 μm, the pore diameter is 1200 to 1800 Å, and the total moisture is 70 to 80%. The functional group is amino (short spacer). The spherical beads are stable at pH 3 to pH 10, and are suitable for storage at 2 to 20° C.

### Rocks

A rock is a solid mass of geological material. In the present invention, the rock may be a source of metal cations.

### Cations

Metal cations used in the method of the present invention include one or more of Mg²⁺, Ca²⁺.

### Rock Composition

According to the present invention, one or more rock types may be applied to soil, as is known in the technique of “enhanced weathering”.

In some embodiments, the rock comprises silica. In some embodiments, the rock comprises or consists of basalt. In some embodiments the rock comprises or consists of olivine.

In some embodiments, the rock comprises carbonates. In some embodiments, the rock comprises limestone.

### Rock Preparation

Prior to application to the soil, rocks that may be used in the method of the present invention may optionally be reduced in size, compared to an initial size, for example to increase rate of enhanced weathering. For example, a boulder may be reduced to a sand, or a pebble may be reduced to a powder or other particulate material typically less than e.g. 5 mm or 1 mm diameter. In some embodiments, the rock is reduced in size by any one or combination of crushing, grinding, sieving, milling, pulverisation, and/or ball milling.

In some embodiments, the rock is particulate and has an average particle size of at least 1 micrometre. In some embodiments, the rock is particulate and has an average particle size of 5 millimetres or less. In some embodiments, the rock is particulate and has an average particle size of 4 millimetres or less. In some embodiments, the rock is particulate and has an average particle size of 1000 micrometres or less. In some embodiments, the rock is particulate and has an average particle size of 750 micrometres or less. In some embodiments, the rock is particulate and has an average particle size of 500 micrometres or less. In some embodiments, the rock is particulate and has an average particle size of 300 micrometres or less. In some embodiments, the rock is particulate and has an average particle size 1 micrometre to 300 micrometres. In some embodiments, the rock is particulate and has an average particle size of 400 micrometres or more. In some embodiments, the rock is particulate and has an average particle size of 500 micrometres or more. In some embodiments, the rock is particulate and has an average particle size of about 500 micrometres to about 4 millimetres. In some embodiments, the rock is particulate and has an average particle size of about 500 micrometres to about 2000 micrometres, or about 500 micrometres to about 1500 micrometres, or about 500 micrometres to about 1000 micrometres, or about 500 micrometres to about 750 micrometres. Any appropriate method for determining particle size may be applied, as known in the art. One simple but effective method for sizing rock particles is sieve analysis, as is well known in the art. There are 2 types of sieve analysis that can be carried out: wet sieving and dry sieving. Wet sieving is suitable for particle sizes from 20 μm up to 3 mm while dry sieving is suitable for particles from 30 μm up to 125 mm.

In some embodiments, the rock is particulate and has an average particle size of at least about 500 micrometres to about 10 millimetres. In some embodiments, the rock is particulate and has an average particle size of at least about 500 micrometres to about 5 millimetres. In some embodiments, the rock is particulate and has an average particle size of at least about 0.5 millimetres to about 4 millimetres. In some embodiments, the rock is particulate and has an average particle size of at least about 500 micrometres to about 2 millimetres. In some embodiments, the rock is particulate and has an average particle size of at least about 500 micrometres to about 1 millimetre.

In some embodiments, the rock is reduced in size to a dust. In some embodiments, the rock is reduced in size to a powder. In some embodiments, the rock is reduced in size to particulates. In some embodiments, the rock is reduced in size to sand.

In some embodiments, the rock is applied to the soil as a dust or sand. In some embodiments, the rock is applied to the soil as a powder. In some embodiments, the rock is applied to the soil as particulate matter.

### Application

Carbonic anhydrase and optionally rock are applied to soil in one aspect of a method of the present invention.

In some embodiments, the Carbonic Anhydrase and rock are applied to the soil simultaneously. Optionally, the Carbonic Anhydrase and rock are pre-formulated together prior to application, or are applied simultaneously from two or more different sources. Optionally, the Carbonic Anhydrase and rock are mixed together prior to application.

In some embodiments, the Carbonic Anhydrase and rock are applied to the soil contemporaneously.

In some embodiments, the Carbonic Anhydrase and rock are applied to the soil at different times. In some embodiments, the Carbonic Anhydrase and/or rock are applied to the soil two or more times, optionally two or more times in a day, a week, a month, a season, or a year.

### Frequency

In some embodiments, the Carbonic Anhydrase and/or rock is applied to the soil once a year. In some embodiments, the Carbonic Anhydrase and/or rock is applied to the soil twice or more a year. In some embodiments, the Carbonic Anhydrase and/or rock is applied to soil over one or more years.

In some embodiments, the Carbonic Anhydrase and/or rock is applied to the soil prior to planting crops.

### Method

The Carbonic Anhydrase and/or the rock can be applied to the soil in different ways.

In some embodiments, the Carbonic Anhydrase and/or rock is sprayed onto soil. In some embodiments, the Carbonic Anhydrase and/or rock is spread onto soil.

In some embodiments, spreading can occur by hand. In some embodiments, spreading can occur by machine. In some embodiments, the spraying can occur by machine. In some embodiments, the Carbonic Anhydrase and/or rock can be dug into the soil, optionally by mixing and/or churning.

Typically, application will be done using machinery over one or more acres. In some embodiments, application will be done using machinery over ten or more acres, fifty or more aces, seventy or more acres, one hundred or more acres, two hundred or more acres, five hundred or more acres, one thousand or more acres, five thousand or more acres, ten thousand or more acres, twenty thousand or more acres. In some embodiments, application will be done using machinery over five or more hectares, ten or more hectares, fifty or more hectares, seventy or more hectares, one hundred or more hectares, two hundred or more hectares, five hundred or more hectares, one thousand or more hectares, five thousand or more hectares, ten thousand or more hectares.

### Soil, Earth, Ground, Ocean

In some embodiments of the present invention, Carbonic Anhydrase and optionally rock are applied to soil. This attenuates, prevents, or reverses soil acidification.

In some embodiments, Carbonic Anhydrase and rock are applied to ground. The ground may or may not comprise soil. For example the ground may be scrubland or rocky ground. The ground may have plant cover, for examples grasses or trees.

In some embodiments, there is not substantial plant cover over the soil. Typically, the soil comprises or consists of bare soil. The rock and Carbonic Anhydrase can therefore directly contact the bare soil.

Soil organic carbon (SOC) is the overall soil carbon content of a soil and may also be generally referred to as total organic carbon (TOC) (the terms may be used interchangeably), and this refers only to the carbon component of the organic matter in the soil. However, fluctuations in soil organic carbon may not necessarily correlate to the same fluctuations in stable soil carbon. Indeed, soils subjected to the treatments and methods may demonstrate minimal increases in TOC, but the percentage of said TOC that is captured in a stable form in the soil or in the fungi proliferating in the soil (i.e., complex polysaccharides, melanin, chitin, lignin, suberin and carotenoid compounds) may increase. The skilled addressee would also understand that changes in TOC and stable carbon in soil as a result of the treatments and methods of the present may take weeks, months or years, and therefore appropriate measurement timeframes must be applied. In one embodiment, the increase in soil organic carbon in a soil comprises an increase in stable carbon in the soil.

The soil carbon may be measured by methods including, but not limited to, dry combustion or elemental tests that may be analysed using, for example, the LECO analysis method, and loss on ignition (LOI) tests that may be analysed using the Walkley-Black method (see, for example, Walkley A, and Black IA (1934) An examination of the Degtjareff method for determining soil organic matter, and a proposed modification of the chromic acid titration method. Soil Science 37, 29-38). To assess the prevalence of different types of carbon on the TOC (i.e. to measure the stable, or “recalcitrant” organic carbon), methods may be employed to fractionate to TOC by, for example, measuring soil respiration or the bulk density of the soil.

In some embodiments, the applying step comprises spraying the Carbonic Anhydrase onto the soil or ground. The Carbonic Anhydrase may be present in a slurry when sprayed, for example a fertilising slurry of the sort often used in agriculture. The slurry may comprise rock as described extensively herein. The Carbonic Anydrase, optionally in the form of a slurry, may be sprayed onto the soil or ground using agricultural equipment for example a spray bar or broadcast spreader.

While the disclosure focuses on solid ground, it can also be applied to large bodies of water such as in an ocean, sea or lake. All technologies of enhanced weathering where finely ground rock is deposited on or in an extensive area, e.g. farm, forest, ocean, lake or sea are contemplated within the scope of the invention.

### Land

In some embodiments, the soil or ground is within agricultural land. In some embodiments, the soil or ground is within arable land. In some embodiments, the soil or ground is within pasture land.

In some embodiments, the soil or ground is within pasture land. In some embodiments, the soil or ground is within waste land. In some embodiments, the soil or ground is within brownfield land. In some embodiments, the soil is within industrial land.

### Depth

The present invention can advantageously retain Carbonic Anhydrase within a desired range of soil depth.

Typically, the Carbonic Anhydrase is retained in the top soil. Methods of measuring the depth of top soil are well known in the art. Typically, the depth of the top soil is measured downwards from the surface of the soil.

In some embodiments, the top soil extends 2 metres or less from the soil surface, and the Carbonic Anhydrase is retained in that region. In some embodiments, the top soil extends 1 metre or less from the soil surface. In some embodiments, the top soil extends 50 centimetres or less from the soil surface. In some embodiments, the top soil extends 30 centimetres or less from the soil surface. In some embodiments, the top soil extends 25 centimetres or less from the soil surface. In some embodiments, the top soil extends 20 centimetres or less from the soil surface. In some embodiments, the top soil extends 15 centimetres or less from the soil surface. In some embodiments, the top soil extends 10 centimetres or less from the soil surface. In some embodiments, the top soil extends 5 centimetres or less from the soil surface.

In some embodiments, the Carbonic Anhydrase is retained substantially on the surface of the top soil. For example, in some embodiments the Carbonic Anhydrase is retained in the top 5 cm of the soil, or the top 1 cm of the soil. In these embodiments, Carbonic Anhydrase remains exposed directly to the surface air and is not buried in the soil.

### Increasing Crop Yield

In the present invention, Carbonic Anhydrase and optionally rock are applied to soil. The Carbonic Anhydrase may be applied to the soil in the form of being immobilised to a seed that is sown into the soil. The Carbonic Anhydrase may be applied to the soil in the form of being immobilised to a plant that is planted into the soil, optionally wherein the Carbonic Anhydrase is immobilised to the plant root. The seed coat and/or plant root may remain in the soil after the plant or a part of the plant is harvested, thereby also retaining the Carbonic Anhydrase in the soil. This attenuates, prevents, or reverses soil acidification, which can increase land productivity. In some embodiments of the invention, land productivity is increased after application of Carbonic Anhydrase and rock to soil.

For example, crop yields in the subsequent year or years can increase. In some embodiments, the crop yield obtained from land treated by application of Carbonic Anhydrase and optionally rock to soil is increased by 5% or more compared to the previous crop yield from the same land. In some embodiments, crop yields are increased by 10% or more, 15% or more, 20% or more, 25% or more.

In other embodiments, the crop yield obtained from land treated by application of Carbonic Anhydrase and optionally rock to soil is increased by 5% or more compared to untreated land. In some embodiments, crop yields are increased by 10% or more, 15% or more, 20% or more, 25% or more.

The time taken to obtain increased crop yield can be over one or more years. In some embodiments, the crop yield is increased in the subsequent year to the first application of Carbonic Anhydrase and rock to soil. In some embodiments, the crop yield is increased two years subsequent to the first application of Carbonic Anhydrase and rock to soil. In some embodiments, the crop yield is increased three years subsequent to the first application of Carbonic Anhydrase and rock to soil. In some embodiments, the crop yield is increased four years subsequent to the first application of Carbonic Anhydrase and rock to soil. In some embodiments, the crop yield is increased five years subsequent to the first application of Carbonic Anhydrase and rock to soil. In some embodiments, the crop yield is increased six years subsequent to the first application of Carbonic Anhydrase and rock to soil.

In some embodiments, the land retains higher productivity after application of Carbonic Anhydrase and rock to soil compared to untreated soil.

In some embodiments, the crop yields are higher in land treated by application of Carbonic Anhydrase and rock to soil compared to crop yields obtained from land treated by application of rock to soil alone. In some embodiments, the crop yields are higher in land treated by application of Carbonic Anhydrase and rock to soil compared to crop yields obtained from land not treated by application of carbonic anhydrase.

### EMBODIMENTS

The disclosure further provides the following numbered embodiments as provided below, which are intended to define further the disclosed technologies but not intended to limit the scope of the invention.
1. A method of sequestering atmospheric carbon, comprising the step of applying Carbonic Anhydrase and rock to soil.
2. The method of embodiment 1, wherein the Carbonic Anhydrase is immobilised to a solid support.
3. The method of embodiment 2, wherein the solid support comprises or consists of:
   plant matter, optionally wherein the plant matter comprises or consists of corn husks and/or cellulose; and/or
   animal matter; and/or
   fibrous materials; and/or
   a bead;
optionally wherein the solid support comprises or consists of waste material.
4. The method of embodiment 2 or 3, wherein the solid support has a procurement cost of $20 or less per kg, $10 or less per kg, $5 or less per kg, $1 or less per kg, or $0.5 or less per kg.
5. The method of embodiment 1, wherein the Carbonic Anhydrase is immobilised to the rock, optionally directly to the rock, optionally by covalent bond or by non-covalent means such as physical adsorption.
6. The method of any preceding embodiment, wherein the Carbonic Anhydrase has a structural modification to the enzyme which is intended to increase retention at a specified depth and/or location in the soil.
7. The method of any preceding embodiment, wherein the Carbonic Anhydrase is retained in top soil; optionally wherein the top soil extends 1 metre or less from the soil surface, optionally 50 centimetres or less, 30 centimetres or less, 25 centimetres or less, 20 centimetres or less, 15 centimetres or less, or 10 centimetres or less.
8. The method of any preceding embodiment, wherein the Carbonic Anhydrase is substantially retained at or around the roots of a plant, optionally in contact with one or more roots.
9. The method of any preceding embodiment, wherein the soil is within:
   agricultural land, optionally wherein the agricultural land is arable land or pasture land;
   waste land;
   brownfield land; or
   industrial land.
10. The method of any preceding embodiment, wherein the Carbonic Anhydrase and rock are applied to the soil simultaneously, contemporaneously or at different times.
11. The method of any preceding embodiment, wherein the rock is a source of metal ions; optionally wherein the metal ions are cations, optionally wherein the cations are any of Mg⁺² or Ca⁺².
12. The method of any preceding embodiment, wherein the rock comprises silica, optionally wherein the rock comprising silica is basalt or olivine.
13. The method of any preceding embodiment, wherein the method comprises the step of reducing the rock in size before application to the soil, optionally wherein the rock is reduced in size by crushing, grinding, sieving, milling, pulverisation, and/or ball milling.
14. The method of any preceding embodiment, wherein the rock is applied to the soil as a dust, powder, or particulate.
15. The method of any preceding embodiment, wherein the Carbonic Anhydrase comprises an animal carbonic anhydrase, a plant carbonic anhydrase, and/or a bacterial carbonic anhydrase; optionally wherein the animal Carbonic Anhydrase comprises bovine erythrocyte carbonic anhydrase.
16. The method of any preceding embodiment, wherein the Carbonic Anhydrase comprises an alpha, beta, delta, gamma, or zeta carbonic anhydrase.
17. The method of any preceding embodiment, wherein the atmospheric carbon is sequestered as a carbonate, optionally as a magnesium carbonate or a calcium carbonate.
18. A sequestered carbon obtained or obtainable by the method of any of embodiments 1-17.
19. A mixture comprising Carbonic Anhydrase and soil, optionally wherein the Carbonic Anhydrase is immobilised to a solid support.
20. A mixture comprising Carbonic Anhydrase and rock, optionally wherein the Carbonic Anhydrase is immobilised to a solid support.
21. A mixture comprising carbonic anhydrase, rock and soil, optionally wherein the Carbonic Anhydrase is immobilised to a solid support.
22. Use of a mixture according to any of embodiments 19-21 to sequester atmospheric carbon.
23. A mixture according to any of embodiments 19-21, for use in sequestering atmospheric carbon.
24. A method of spraying an enzyme slurry or otherwise coating rock in a way that is compatible with agricultural equipment, e.g. a spray bar on a piece of broadcaster equipment.
25. Carbonic anhydrase enzyme within a cell and bound to rock, such as via adsorption to rock particles of a cell that expresses Carbonic Anhydrase.

### EXAMPLES

### Summary: Carbon Capture Product to Accelerate Enhanced Weathering.

Test suitability of commercially-available Carbonic Anhydrase (CA) enzyme for accelerated enhanced weathering using agriculturally acceptable solid carriers. Test activity and stability of the free enzyme under industrially relevant conditions (e.g. Temperature 4-20° C., ambient CO₂, neutral and basic pH). Using commercial Carbonic Anhydrase enzyme, perform industrially relevant bead screens (e.g. basalt, vermiculite, perlite) for stability and activity.

### BACKGROUND

—**Basalt.** In geology basalt reacts with CO₂ from rain water to produce magnesium and calcium salts, such as, bicarbonate to store this mineral in the ground for millions of years. Basalt is mainly composed of SiO2 (45-52%), with CaO (5-10%), MgO (9.7%) and Na₂O (5.3%) plus many other metal oxides such as Al₂O₃ (13.1%) and Fe₂O₃ (13.8%). [Basalt is negatively charged.]
—**Advanced weathering.** Accelerate this natural process by spreading raw and processed basalt on fields to capture and store CO₂.
—**Carbonic anhydrase.** This is found in nature and converts CO₂ and H₂O into carbonic acid and protons, we want to utilise this enzyme to accelerate the advanced weathering phenomena by coating the enzymes directly on basalt rocks. It [converts 1,000,000 CO₂ molecules per second]. This enzyme has been shown to bind to silica gel previously—see https://pubmed.ncbi.nlm.nih.gov/18584683/.
—**CO₂ produced per year.** Across the world 93,000,000 barrels of oil are used per day (1 barrel=200 L). CO₂ emissions equate to 36.8 billion tonnes per year. We need to remove 1 billon tonnes per year to help slow down global warming.
—**Alternative rocks.** Other rock types or minerals can also exhibit favourable properties for this application—see (https://en.wikipedia.org/wiki/List_of_rock_types). However most researchers are focused towards basalt.
—**Bead screen.** Initially we planned to perform a bead screen using a variety of agricultural beads, including basalt, vermiculite and perlite, plus and minus coating agents to improve stability. However looking into the feasibility of the project in more detail we realised it only makes sense to coat the enzyme directly onto basalt for the current application. Veolia have supplied two types of basalt, *raw* and *processed*. The latter is smaller and has a higher water content by around 30-40% v/w.

### Key Findings

CA from a commercial source is highly concentrated and active>Sigma Carbonic Anhydrase extracted from cows>bovine serum.

CA is able to turn over 4-nitrophenyl acetate or CO₂.

CA is able to accelerate CO₂ conversion using either NaCl, MgCl2, CaCl2 or basalt.

CA is able to bind and remain stable for up to 1 month at RT to either basalt, silica gel with or without fragmentation.

CA immobilised on basalt is stable and remains active over 4 uses.

CA from a commercial source loads 10 μL of enzyme onto 1 g of raw or processed basalt in 30 mins at RT in tap water.

Carbonic Anhydrase should be diluted down to 50 L in tap water and then sprayed onto basalt directly on the field.

Carbonic Anhydrase can get washed off basalt rocks in soil so careful attention must be paid to how it interacts with soil.

Carbonic Anhydrase immobilised on silica gel is able to turn over CO₂ and react with “sea water” in flow leading to a sea-based product.

### Example 1: Exemplary Immobilisation Soil Test Protocol

Take 50 L of 1×PBS buffer in a >50 L container. Add the buffer reagent to the >50 L container.
Add 250 g of Silica Gel
Add 6 g of enzyme
Agitate/mix with a stirrer for 4 hrs at room temp (25-30° C.)
Apply directly to the soil as a wet solution.

### Example 2. Carbonic Anhydrase (CA) Obtained from Residual Blood from Meat Products

Bovine blood typically contains 7-15% w/v of CA. In this example, CA was obtained from residual blood from grocery store steaks, from Amazon and Sainsbury's. In its unpasteurised form, milk contains CA. However, the pasteurisation process was expected to denature the majority, if not all, of the Carbonic Anhydrase. Therefore, pasteurised milk was used as a negative control in these experiments.

Method: 20 μL of blood solution was extracted from steaks obtained from Amazon and Sainsburys and placed into 180 μL water, then performed a 1 in 2 serial dilution. 10 μL of this liquid was used in a 4-nitrophenol acetate (4-NPA) assay. The 4-NPA assay involves the hydrolysis of 4-NPA by CA, and is described, for example in Shank et al. (2006) Chemical Biology & Drug Design 68 (2) 113-119. Specifically, 4-NPA, which is colourless, is hydrolysed to 4-nitrophenol (4-NP), which is yellow, and acetate. This colour change provides a spectrophotometric optical density readout.

Specifically, the 10 μL of and placed into 50 μL of 4-NPA mastermix in a 384-well plate. The absorbance was scanned at 400 and 500 nm.

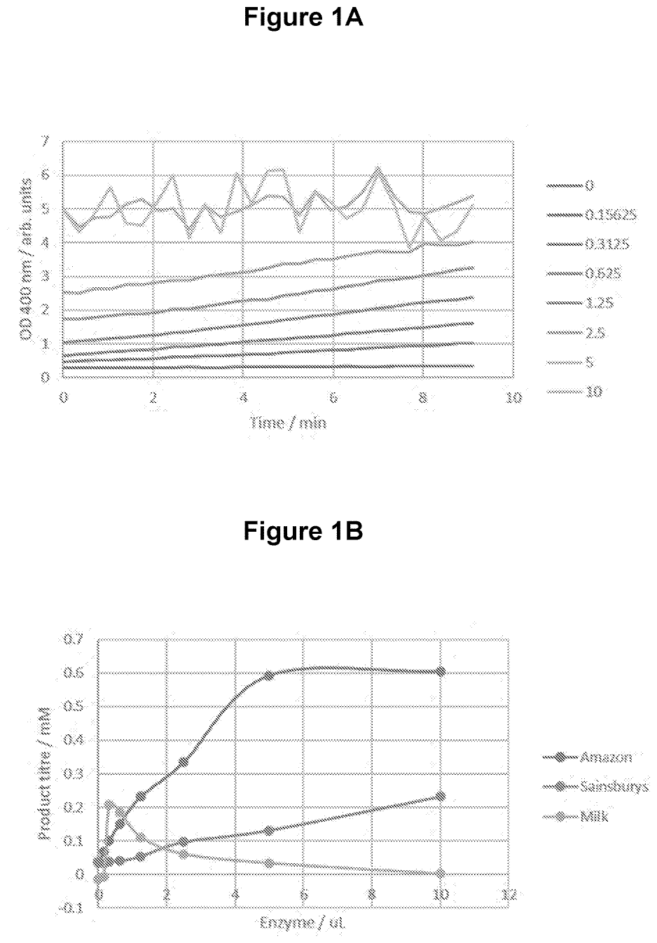

Outcome: CA from both Amazon and Sainsbury's was able to convert 4-NPA to 4-NP. As expected, pasteurised milk contained no active CA. FIG. 1A shows outcome of the 4-NPA assay when serial dilutions of the blood solution obtained from the Amazon steak were used. FIG. 1B shows the product titre obtained from CA obtained from Amazon steak, Sainsbury's steak, or pasteurised milk.

This example demonstrates that active CA can be obtained from an animal or part thereof, for example from the blood of the animal.

### Example 3. CA Obtained from Animal Blood has Comparable Activity to Commercial CA

This example shows that commercially-available CA, bovine serum, and blood from steak and minced beef show 4-NPA activity.

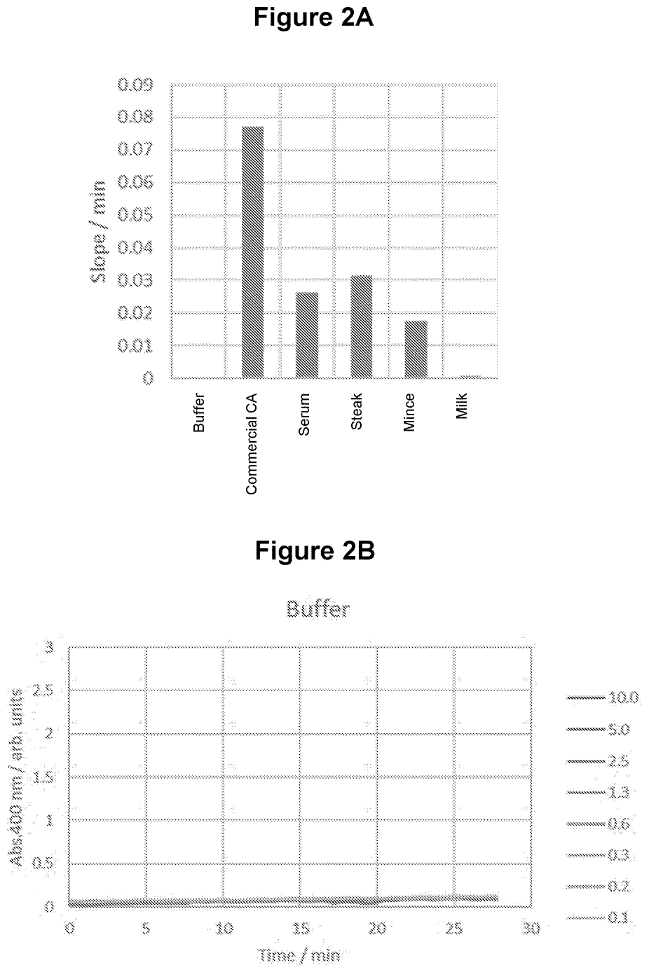

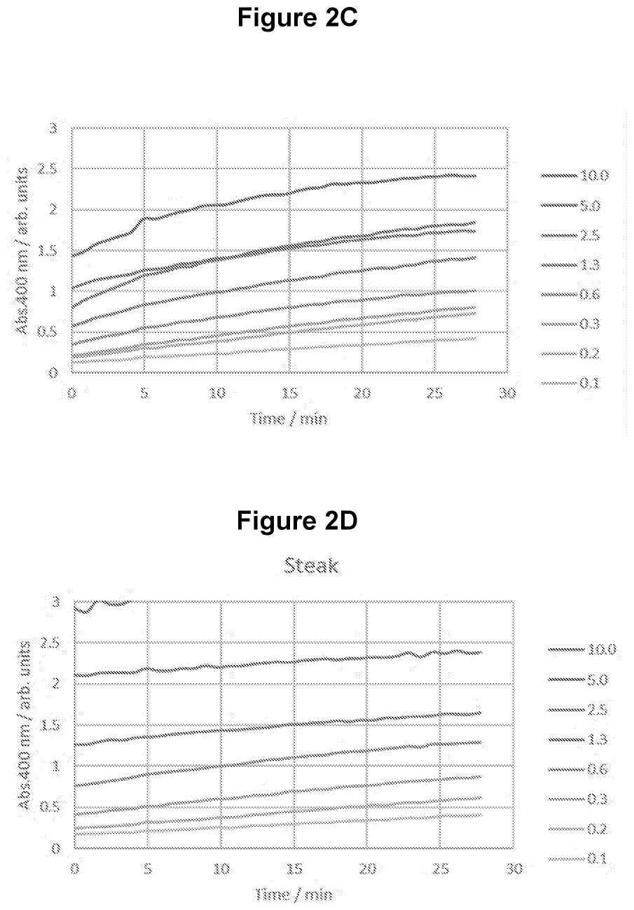

The 4-NPA assay described in Example 2 was repeated. Specifically, 1 mL of each CA source, underwent a 1 in 2 serial dilution, into a 96 deep well plate. Next, 10 μL of solution was added to 50 μL of 4-NPA mastermix in a 384-well plate. The profiles were plotted and the slopes generated. FIG. 2A is a plot of activity slopes of the stated CA source assayed. FIG. 2B shows the activity kinetic plot of the buffer only, as negative control. FIG. 2C shows activity kinetic plot for commercial CA with varying enzyme amount. FIG. 2D shows activity kinetic plot for CA obtained from blood sample obtained from steak. Note, steak had a high background due to particle scattering, therefore the 5 μL plot was used to compare side-by-side.

Next, the thermal melting profile of the free enzymes was assessed.

Method: Commercial CA was diluted 1 in 5 into tap water, and steak blood was diluted 1 in 2 into tap water. The enzyme solution was transferred to 100 μL to 8×PCR tubes and heated at the stated temperature for 10 mins. Next, 10 μL of solution was added to 50 μL of 4-NPA mastermix in a 384-well plate and the 4-NPA assay as described in Example 2 was performed. The normalised profiles were plotted and the slopes generated.

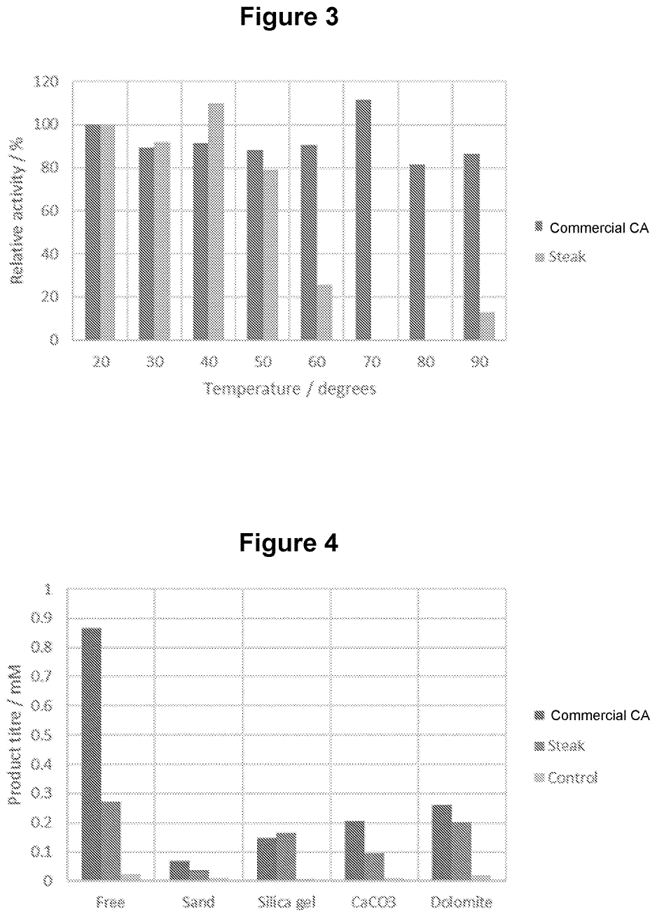

Results: FIG. 3 shows steak CA enzymes denature between 50 and 60 degrees, whilst the commercially-available CA did not unfold at all.

### Example 4. Immobilising Enzyme to Different Solid Supports

This example demonstrates that CA from various sources is able to bind and turn over product on sand, silica gel, chalk and dolomite.

Methods: 200 mg of the stated material was added to 100 μL of the stated crude enzyme. The enzyme was allowed to immobilise for 30 mins by shaking at 1250 r.p.m. at room temperature. The supports were washed by performing 5× wash-centrifugation cycles (except for the free enzyme controls which were not washed). Next, a 4-NPA activity assay was performed by adding 1 mL 4-NPA mastermix and shaking at 1250 r.p.m. for 30 minutes, isolating 50 μL of supernatant and transferring to a 384-well plate. The amount of product formed was plotted.

Results: FIG. 4 shows CA from both sources was able to bind and turn over 4-NPA on sand, silica gel, chalk and dolomite. The commercial enzyme was added in about 5× excess, whilst the blood ratio was more efficient with almost quantitative loading.

### Example 5. Carbonate Formation

This example demonstrates that CA obtained from bovine blood is able to capture and convert CO₂ into sodium carbonate and or calcium carbonate using sea water.

Method: 1 g of silica gel with or without 200 μL of Amazon CA was weighed out and placed in a 50 mL falcon tube and left to immobilise for 30 minutes. 10 mL sea water containing 1M tris base at pH 10.5 was then added to control the pH shift. 1 mL of this mix was extracted and 100 μL of concentrated HCl was added. The falcon tube was heated to 50 degrees and 3× cardice (i.e., dry ice) cubes were added. After 10 minutes 1 mL was extracted and 100 μL of concentrated HCl was added.

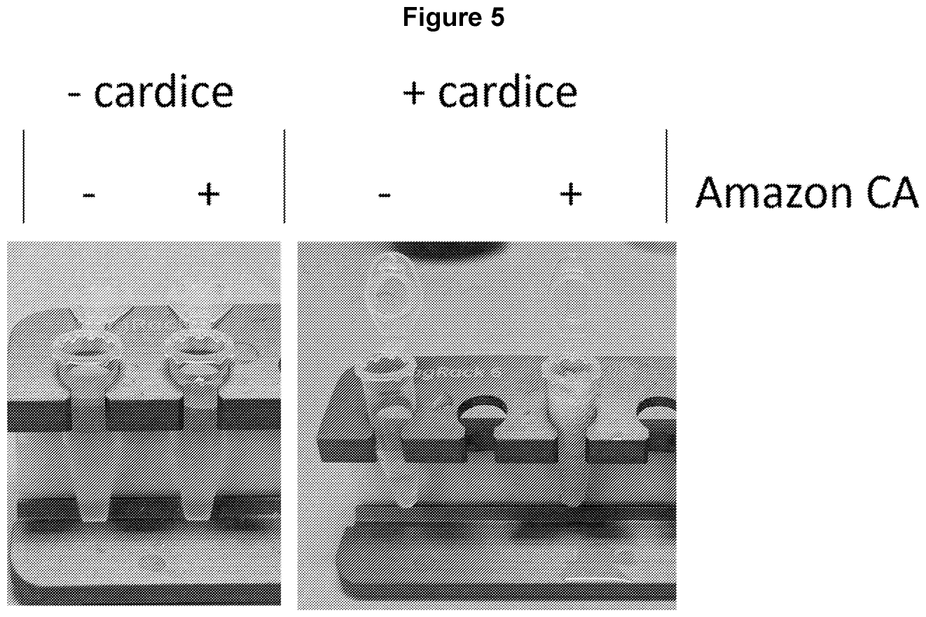

Results: FIG. 5 shows extensive bubble formation for CA obtained from bovine blood after the addition of cardice, indicating efficient CO₂ capture and conversion to sodium carbonate or calcium carbonate.

### Example 6

In 6×50 mL falcon tubes, either 0, 1, or 10 mL of crude CA from a commercial source was added to 0.5 g of raw “R” or processed “P” basalt, supplemented with 0.1 M NaPi pH 7.0 (25 mL). All volumes were equal. Next, the falcon tubes were placed in a water bath set to 50 degrees, after 5 minutes single blocks of cardice were added and the colour change monitored.

After 10 minutes, 50 μL was extracted and scanned the absorbance in a 384-well plate using the plate reader.

Pictures and absorbance profiles did not show the corresponding change in acid, potentially the basalt interacts with the indicator to corrupt the readout. Also, no apparent change in basalt mass was observed.

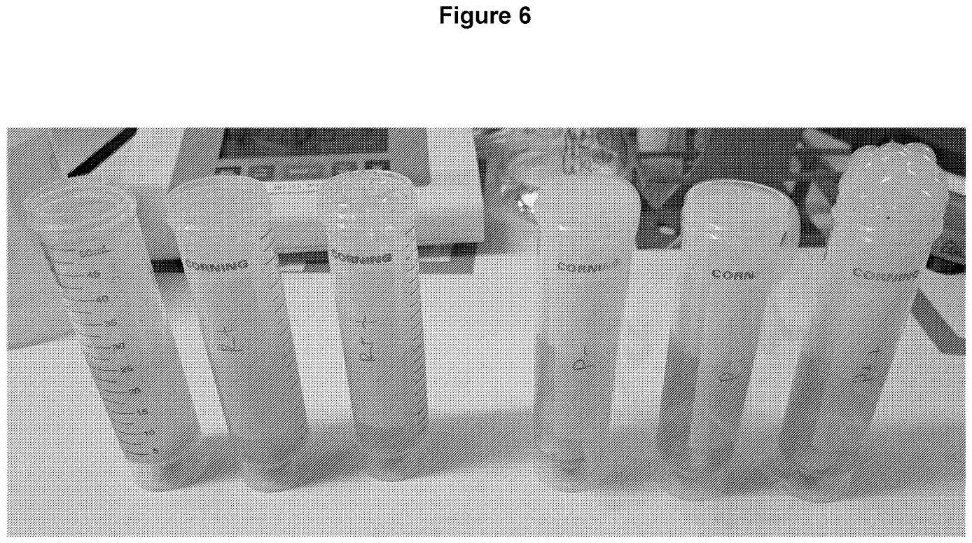

FIG. 6 shows that bubbles formed when either raw “R” or processed “P” basalt was provided to the mix.

### Example 7

This example demonstrates that CA isolated from blood can be immobilised on CaCO3 and improve its thermal stability.

Method: 0.25 mg of CaCO3−/+200 μL of steak blood CA was added to a 1.5 mL vial and left to immobilise for 30 minutes. The immobilised CA was then washed in water by performing 5× wash-centrifugation cycles. Next, the immobilised CA was moved into 100 μL PCR tubes and heated to the stated temperature for 10 minutes. Next, 100 μL of 4-NPA mastermix was added in a 384-well plate and mixed for 10 mins shaking at 1250 r.p.m., then extracted 50 μL supernatant. The absorbance was scanned. The normalised profiles were plotted and the slopes generated.

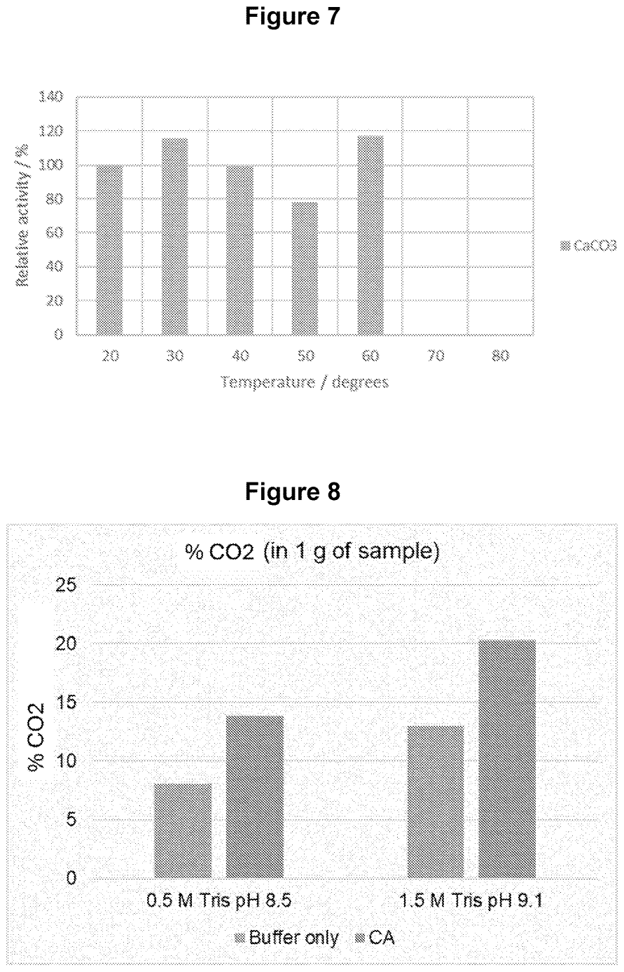

FIG. 7 shows CA can be stabilised on CaCO3 (chalk) and turn over 4-NPA product up to 60° C.

### Overview of Examples 8 to 18

The following examples show that the CO₂ sequestration is effective for different sources of basalt. The use of a calcimeter provides a reproducible method for verification.

### Example 8

This example explains how measurements using a Calcimeter in subsequent examples were conducted.

Method: This analysis was performed using FOGL Bench-top Soil Calcimeter™ from bd Inventions. 5 g of sample was weighed into the sample container. 6 mL of 50% HCl (concentrated HCl diluted 1:1 v/v using double distilled water) was added into the HCl container of the instrument. The sample weight was registered as 5 g and the measurement was initiated by pressing ‘start’ on the software. The lid was closed and all connections were ensured to be secure and airtight. The sample was mixed for 10 s and rested. The resulting change in partial pressure due to CO₂ was measured by the instrument and reported as the % CO₂ equivalents of CaCO3.

### Example 9

This example demonstrates free enzyme activity in water.

Method: 0.25 μl of a CA obtained commercially, 10 mL of water and 0.5 mL of 5 M CaCl2 were mixed. A negative control reaction was also set up, where water was used instead of enzyme. Each reaction was incubated at 60° C. for 5 min in the water bath. Next, one block of equal sized dry ice was added into each tube and the experimentalist waited until the bubbling had stopped to close the tubes and incubate the tubes at 30° C. for 5 min in a shaker, for the tubes were centrifuged for 5 min at 4,500×rpm. The supernatant was decanted and the pellet was resuspended in 1 mL of ddH2O. The % CO₂ for the 1 g sample was then measured using the Calcimeter.

TABLE 1
Variation of % CO2 equivalents for free enzyme reaction.

| Sample | % CO2 |
|---|---|
| Water − ve ctrl_R1 | 0.10 |
| Water − ve ctrl_R2 | 0.12 |
| Enzyme + ve ctrl_R1 | 0.24 |
| Enzyme + ve ctrl_R1 | 0.22 |

No pellet had formed after the reaction, most likely because the dry ice added was not sufficient for the reaction to proceed within the limited time.

### Example 10

This example demonstrates the effect of buffer on the free enzyme reaction.

Method: 2 mL of a CA obtained commercially or, for negative control, buffer only, 10 ml of 1.5 M Tris-HCl PH 9.1 or 0.5 M Tris-HCl pH 8.5 (the experiment was performed using each type of buffer) and 0.5 mL of 5 M CaCl2 were mixed in a 50 mL Falcon tube. The tubes were then incubated in a water bath set to 60° C. for 15 min in a fume hood. A small piece of dry ice was placed into each 50 mL Falcon tube. The reaction was allowed to proceed until the dry ice fumes stopped being released from the tubes. The tubes were incubate in the shaking incubator for 5 min (set to 37° C.). The tubes were then centrifuged for 1 min at 4,300×rcf. The supernatant was decanted from the tubes. The pellets were resuspended in 1 mL of ddH2O. The calcimeter was set to measure 1 g of material; 1 mL of resuspended pellet from 2 h was pipetted into the Calcimeter's Duran bottle and determine the carbonate content using the Calcimeter.

The outcome of the experiment is shown in FIG. 8, where it was found that 1.5M Tris HCl buffer pH 9.1 is better for free carbonic anhydrase reaction.

### Example 11

This example demonstrates the effect of CaCl2 on free enzyme activity.

Method: Three tubes containing 0.25 μl of a commercially obtained CA and 10 ml of 1.5 M Tris pH 9.1 were set up. Either 0.1 mL of 5 M CaCl2 (Tube 1), 0.5 mL of 5 M CaCl2 (Tube 2) or 1 mL of 5 M CaCl2 (Tube 3) were added to each tube, which were then incubated at 60° C. for 5 min in a water bath in a fume hood. One block of equal sized dry ice was added into each tube and the experimentalist waited until the bubbling had stopped before closing the tubes and incubating the tubes at 30° C. for 5 min in the shaker. The tubes were then centrifuged for 5 min at 4,500×rpm. The supernatant was decanted and the pellet was resuspended in 1 mL of ddH2O. The % CO₂ for the 1 g sample was measured using the Calcimeter.

TABLE 2
Variation of % CO2 equivalents with CaCl2
concentration for free enzyme reaction.

| Sample | % CO2 |
|---|---|
| 0.1 mL of 5M CaCl2 | 2.33 |
| 0.5 mL of 5M CaCl2 | 11.03 |
| 2 mL of 5M CaCl2 | 5.89 |

It was found 0.5 M CaCl2 was the optimum concentration to achieve the highest CO2 sequestration using free enzyme.

### Example 12

This example compares of carbonic anhydrase performance on Silica gel or basalt in buffered reaction.

Method: 1 mL of CA obtained commercially or ddH2O were mixed with 10 g of either Remin basalt (Rb), Duntilland basalt (Db) or 2.5 g of Silica gel (SG) in a 50 mL Falcon tube. 10 mL of 1.5 M Tris pH 9.1 buffer was added to each tube. The tubes were incubated on the rotor for 20 min at room temperature (RT). The immobilised CA was washed twice by adding 10 ml of ddH2O. 10 mL of 1.5 M Tris-HCl PH 9.1 and 0.5 mL of 5 M CaCl2 were added to the immobilised CAs. The tubes were incubated in the water bath set to 60° C. for 15 min in the fume hood. A small piece of dry ice was added to each tube. The reaction was allowed to proceed until the dry ice fumes stopped being released from the tubes. The tubes were incubated in the shaking incubator for 5 min (set to 37° C.). The tubes were then centrifuged for 1 min at 4,300×rcf. The supernatant was decanted from the tubes and the carbonate content was measured using the Calcimeter.

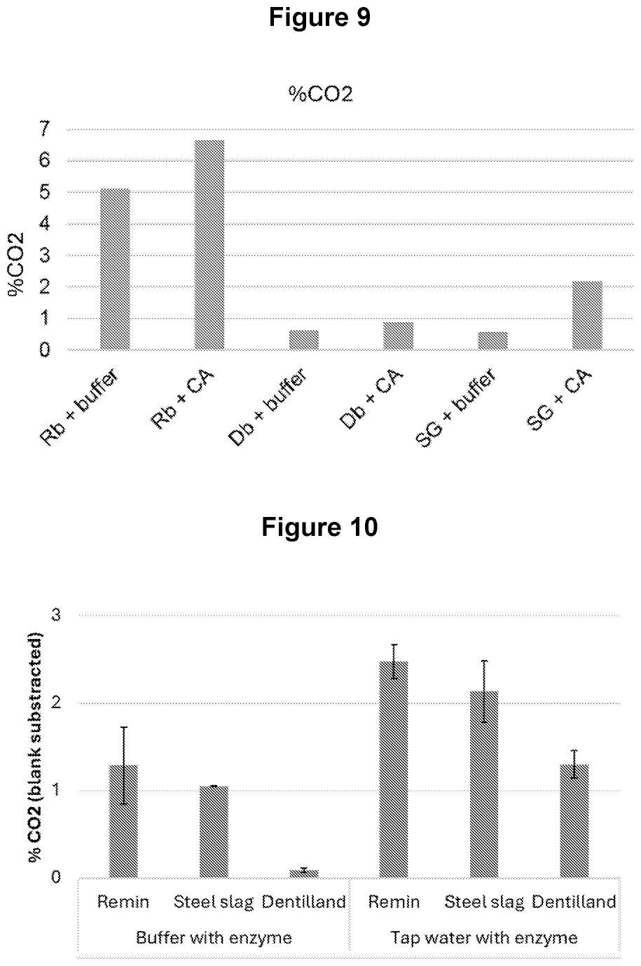

The outcome of the experiment is shown in FIG. 9, which shows that Remin basalt (Rb) performed comparably to silica gel (SG) in terms of CO₂ sequestration.

### Example 13

This example compares the effect of buffer and tap water on the CA reaction.

Method: 1 mL of CA obtained commercially was mixed with 10 g of either Remin basalt (Remin) or Duntilland basalt (Duntilland) or Tata steel slag (Steel slag) in a 50 mL Falcon tube. The Dentilland basalt and Tata steel slag both had particles with broad size up to 4000 micrometre. 10 mL of 1.5 M Tris pH 9.1 buffer was added to each tube. The tubes were incubated on a rotor for 20 min at room temperature (RT). The immobilised CA was washed twice by adding 10 mL of ddH2O. 10 mL of 1.5 M Tris-HCl PH 9.1 or 10-mL tap water was added to the immobilised CAs. 0.5 mL of 5 M CaCl2 was added to all the Falcon tubes. The tubes were incubated in the water bath set to 60° C. for 15 min in the fume hood. A small piece of dry ice was added to each tube. The reaction was allowed to proceed until the dry ice fumes stopped being released from the tubes. The tubes were incubated in the shaking incubator for 5 min (set to 37° C.). The tubes were centrifuged for 1 min at 4,300×rcf. The supernatant was decanted from the tubes and the carbonate content was measured using the Calcimeter.

The outcome of the experiment is shown in FIG. 10, which shows that tap water had considerable impact on CO2 sequestration. This is most likely due to the presence of divalent cations in the tap water, which are known to enhance carbonic anhydrase activity. Furthermore, steel slag was shown to have comparable performance to that of Remin basalt.

### Example 14

This example demonstrates onboarding of basalt sourced from Crag Mill.

Method: 1 mL of CA obtained commercially was mixed with 10 g of either Remin basalt (Remin) or Duntilland basalt (Duntilland) or Crag Mill basalt in a 50 mL Falcon tube. The Dentilland basalt and Crag Mill basalt both had particles with broad size up to 4000 micrometre. 10 mL of tap water was added to each tube. The tubes were incubated on the rotor for 20 min at room temperature (RT). The immobilised CA was washed twice by adding 10 ml of tap water. 10 mL of tap water and 0.5 mL of 5 M CaCl2 were added to each tube. The tubes were incubated in the water bath set to 60° C. for 15 min in the fume hood. A small piece of dry ice was added to each tube. The reaction was allowed to proceed until the dry ice fumes stopped being released from the tubes. The tubes were incubated in the shaking incubator for 5 min (set to 37° C.). The tubes were then centrifuged for 1 min at 4,300×rcf. The supernatant was decanted from the tubes and the carbonate content was measured using the Calcimeter.

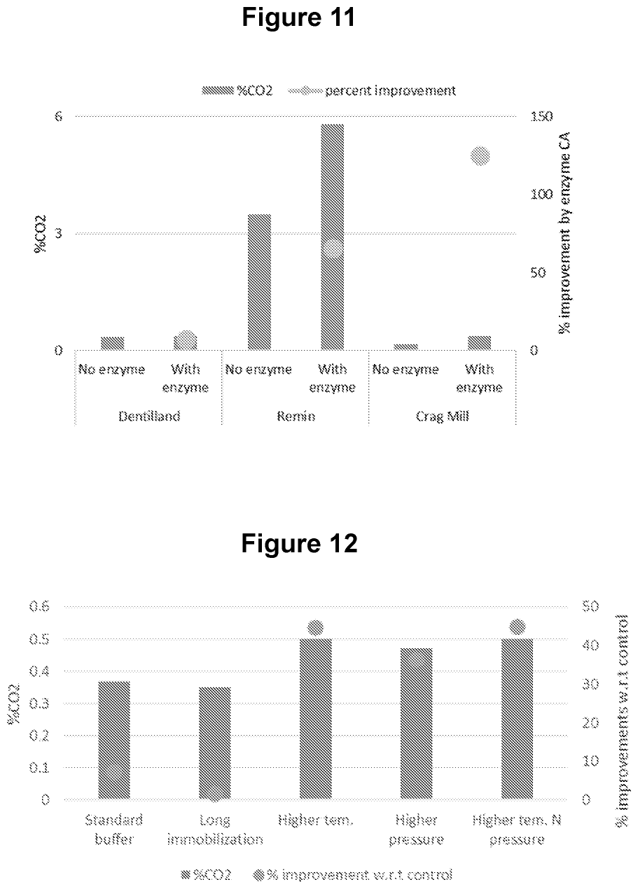

The outcome of the experiment is shown in FIG. 11, which shows that the absolute % CO2 equivalent captured using Crag Mill basalt is comparable to that captured using Dentilland basalt. However, the percentage improvement attained by adding enzymes to the basalt is highest at 125% compared to 67% and 7.5% for Remin basalt and Dentilland basalt, respectively.

### Example 15

This example demonstrates the effect of temperature, pressure, longer immobilization, and combination of increased pressure and temperature on the performance of CA immobilized on Dentilland basalt.

Method: 1 mL of CA obtained commercially was mixed with 10 g of Duntilland basalt (particle size up to 4000 micrometre) in each of 50 mL Falcon tubes 1, 2, 3, and 4. 10 mL of tap water was added to each tube. Tubes 1, 2, and 4 were incubated on the rotor for 20 min at RT, while tube 3 was incubated for 24 h. The immobilised CA was washed twice by adding 10 mL of tap water. 10 mL tap water and 0.5 mL of 5 M CaCl2 were added to the immobilised CAs. Each of the tubes were incubated in the water bath set to 60° C. for 15 min in the fume hood. 2.5 g of dry ice was added to each tube. The reaction was allowed to proceed until the dry ice fumes stopped being released from tubes 1 and 3. For tubes 2 and 4, dry ice fumes were trapped in the falcon to increase the pressure. Tubes 2 and 3 were incubated at 37° C., while tubes 1 and 4 were incubated at 47° C. for 5 minutes in a shaker incubator. The tubes were centrifuged for 1 min post reaction at 4,300×rcf. The supernatant was decanted from the tubes and the carbonate content was measured using the Calcimeter.

The outcome of the experiment is shown in FIG. 12, which shows that the kinetic process conditions such as temperature, pressure did not influence the reaction performance of immobilized CA. Additionally, longer immobilization did not increase the enzyme loading or the CO2 capture.

### Example 16

This example demonstrates the effect of long-term CO2 exposure.

Method: 1 mL of CA obtained commercially or 1 mL water for control reaction was mixed with 5 g of Duntilland basalt (particle size up to 4000 micrometre) in a 10 mL packed bed reactor.

10 mL of tap water was added to each reactor. The reactors were incubated for 60 min at room temperature (RT). CO2 gas was fed into the reactors for 23 h at 0.5 bars using a pressure regulator. To avoid dehydration by CO2, the CO2 was bubbled through water. The basalt was decanted into a clean Petri dish and the carbonate content was measured using the Calcimeter.

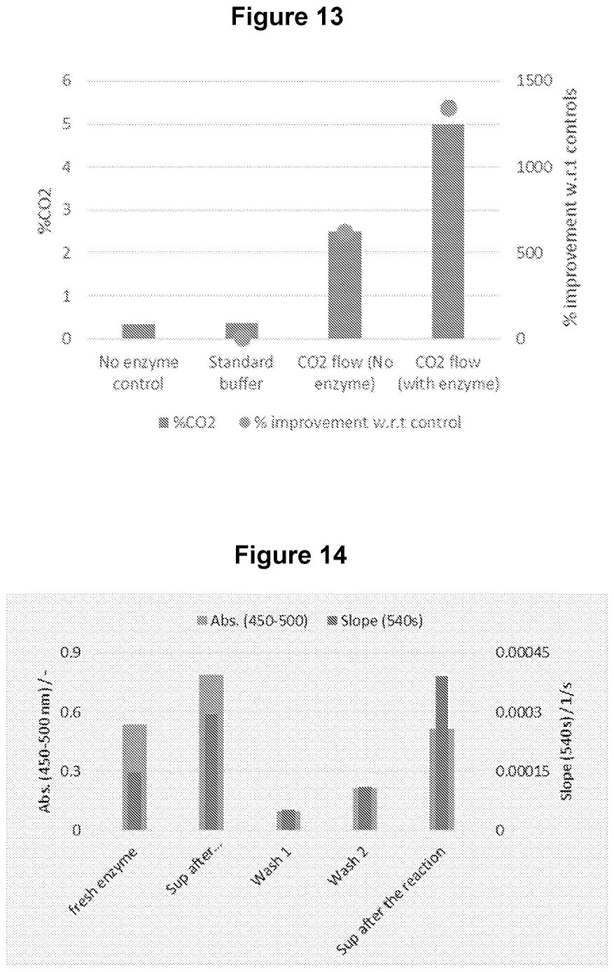

The outcome of this experiment is shown in FIG. 13, which shows that the flow of CO2 increases the residence time and the mass transfer co-efficient. This improves the CO2 sequestration by 13×.

### Example 17

This example demonstrates immobilization efficiency and leaching of CA on Dentilland basalt. Method: 1 mL of CA obtained commercially was mixed with 10 g of Duntilland basalt in a 50 mL Falcon tube. 10 mL of tap water was added to the tube. The tube was incubated on the rotor for 20 min at room temperature (RT). The immobilised CA was washed twice by adding 10 mL of tap water. The supernatant post immobilization and the wash fractions were collected for later analysis. 10 mL of tap water and 0.5 mL of 5 M CaCl2 were added to the immobilised CAs. The reaction tube was incubated in the water bath set to 60° C. for 15 min in the fume hood. 1 g of dry ice was added to the tube. The reaction was allowed to proceed until the dry ice fumes stopped being released and the tube was incubated at 37° C. for 5 minutes in a shaker incubator. After the reaction, the tube was centrifuged for 1 min post reaction at 4,300×rcf and the supernatant fraction was collected. The enzyme activity of all the fractions was determined using 4-NPA assay and compared with the fresh enzyme of same dilution. Carbonic anhydrase is bound to basalt using non-specific ionic interactions. The outcome of the experiment is shown in FIG. 14.

### Example 18

This example demonstrates the results obtained from pre-trials in open field.

Method: Water permeable membrane was spread on grass land. 36 plots of 2 m by 2 m were measured and marked with ropes. 16 kg of Dentilland basalt or Remin basalt was added to 50 L water and the content was poured in the centre of the grid. The basalt was spread over the grid to form a layer. The bio-actives shown in Table 3 were sprayed at the required concentration of the respective grid. Samples were taken weekly on days on which precipitation did not occur. Approximately 50 g of basalt was scooped into a 50 mL falcon for sampling. The in situ pH probe was used to measure the pH in parallel during sampling. The residual enzyme activity was measured using the 4NPA assay and the carbonate content was measured using a Calcimeter.

TABLE 3
The different bio actives used in the pre-trial.

| Posi- | | | | | | pH of |
|---|---|---|---|---|---|---|
| tion | Num- | Basalt | | CA | | plot on |
| ID | ber | source | CA source | amount | | day 12 |
| A1 | 1 | Dentilland | CA obtained | 50 | mL | 6.3 |
| | | | commercially | | | |
| A2 | 2 | Dentilland | CA obtained | 5 | mL | 6.3 |
| | | | commercially | | | |
| A3 | 3 | Dentilland | CA obtained | 0.5 | mL | 6.1 |
| | | | commercially | | | |
| A4 | 4 | Dentilland | CA obtained | 0.05 | mL | 6.2 |
| | | | commercially | | | |
| A5 | 5 | Dentilland | CA obtained | 0.005 | mL | 5.1 |
| | | | commercially | | | |
| A6 | 6 | Dentilland | None | 0 | mL | 6 |
| B1 | 7 | Dentilland | CA obtained | 50 | mL | 7.5 |
| | | | commercially | | | |
| B2 | 8 | Dentilland | CA obtained | 5 | mL | 6.5 |
| | | | commercially | | | |
| B3 | 9 | Dentilland | CA obtained | 0.5 | mL | 6.2 |
| | | | commercially | | | |
| B4 | 10 | Dentilland | CA obtained | 0.05 | mL | 6.2 |
| | | | commercially | | | |
| B5 | 11 | Dentilland | CA obtained | 0.005 | mL | 5.8 |
| | | | commercially | | | |
| B6 | 12 | Dentilland | None | 0 | mL | 6.2 |
| C1 | 13 | Dentilland | Chicken serum | 100 | mL | 6.5 |
| C2 | 14 | Dentilland | Potato mash | 5x | potato | 6.5 |
| C3 | 15 | Dentilland | Bakers' yeast | 1 | pot | 6.3 |
| C4 | 16 | Dentilland | Bakers' yeast + | 1 pot + 1 | | 4.5 |
| | | | glucose | bag | | |
| C5 | 17 | Dentilland | Brewers' yeast | 1 | pot | 6.3 |
| C6 | 18 | Dentilland | Brewers' yeast + | 1 pot + 1 | | 6 |
| | | | glucose | bag | | |
| D1 | 19 | Dentilland | Bacterial spores | 50 | mL | 6.3 |
| D2 | 20 | Dentilland | Compost starter | 1 | bag | 5.7 |
| D3 | 21 | Dentilland | Yakult | 1 | L | 6.5 |
| D4 | 22 | Dentilland | Eggs | 1 | L | 6.3 |
| D5 | 23 | Dentilland | Raw goats milk | 1 | L | 6.2 |
| D6 | 24 | Dentilland | Mince beef Tescos | 1 | bag | 6.5 |
| E1 | 25 | Dentilland | Mince pork Tescos | 1 | bag | 6.5 |
| E2 | 26 | Dentilland | Kafir | 1 | bag | 6.2 |
| E3 | 27 | Dentilland | Actimel | 8x | bottles | 6.4 |
| E4 | 28 | Dentilland | Actimel plus | 8x | bottles | 6.3 |
| E5 | 29 | Dentilland | Raw cows milk | 1 | L | 6.5 |
| E6 | 30 | Dentilland | Grass | 1 | kg | 6.6 |
| F1 | 31 | Dentilland | CA obtained | 50 | mL | 6.9 |
| | | | commercially | | | |
| F2 | 32 | Dentilland | CA obtained | 5 | mL | 6.7 |
| | | | commercially | | | |
| F3 | 33 | Dentilland | Soil | 8 | kg | 6.3 |
| F4 | 34 | Dentilland | Soil | 16 | kg | 6.3 |

| | | | | | |
|---|---|---|---|---|---|
| F5 | 35 | Dentilland | Empty then added | — | 6.3 |

100 g CaCO3 after
2 weeks

| | | | | | |
|---|---|---|---|---|---|
| F6 | 36 | Dentilland | Left in tub | — | 6.3 |

| | | containing rain | | | |
|---|---|---|---|---|---|
| | | water | | | |
| Alpha | Remin | None | — | | 6.2 |
| Beta | Remin | Yakult | 8x | bottle | 5.3 |
| Gamma | Remin | Bakers' yeast | 8x | sachet | 6.2 |
| Delta | Remin | Kafir | 3x | bottles | 5.9 |

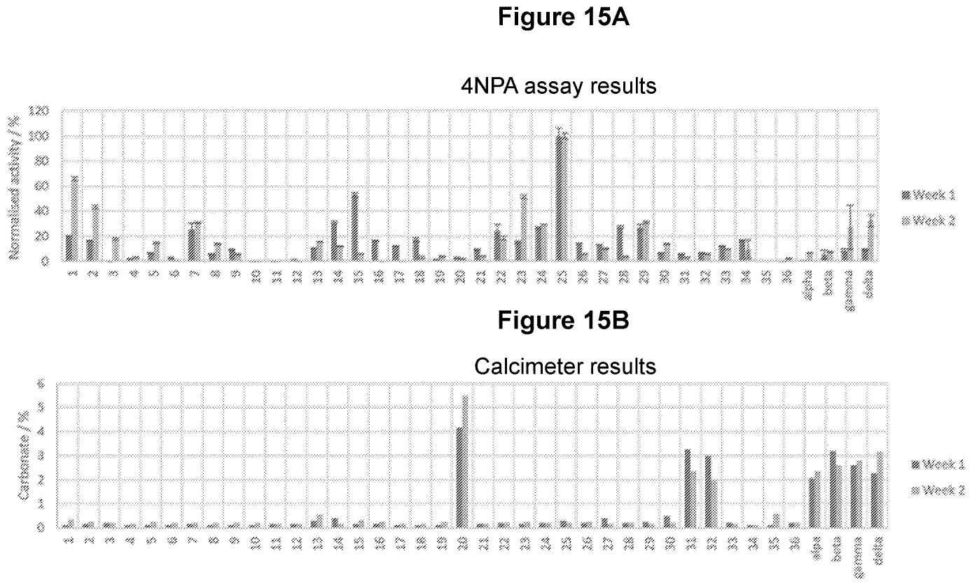

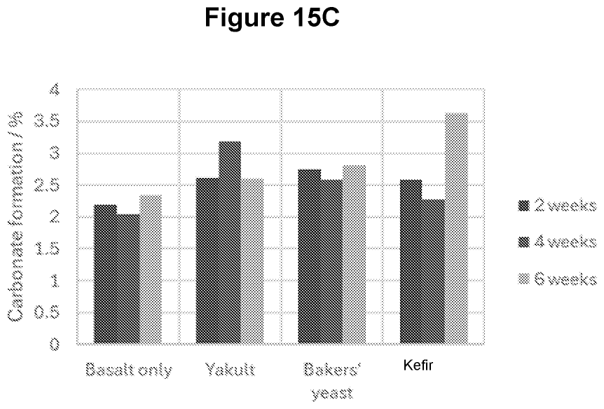

The outcome of the experiment is shown in FIGS. 15A, 15B and 15C.

The pH measurements of the soil are shown in Table 3. These pH measurements were obtained when the soil temperature was 8.3° C., when there had been no precipitation for at least 24 hours, and were taken on day X12 after application of each CA source to the respective plot. Corresponding alpha, beta, gamma and delta plots, with Remin basalt, measured on day 12, were pH 6.2, 5.3, 6.2, and 5.9, respectively. The four control plots (without basalt and enzyme), measured on day 12, were pH 6, 6, 6.1, and 6.2.

It was observed the CA obtained commercially along with Kefir, Yakult and baker's yeast show relative increase in the CO2 equivalent measured using a Calcimeter, which indicated the presence of active CA and improved CO2 sequestration.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1. A method of sequestering atmospheric carbon, comprising the step of applying Carbonic Anhydrase to soil.

2. A method according claim 1, wherein the step of applying the Carbonic Anhydrase to the soil compromises applying the Carbonic Anhydrase and rock to the soil.

3. The method of claim 1, wherein the Carbonic Anhydrase is immobilised to a solid support.

4. The method of claim 3, wherein the solid support comprises or consists of:
plant matter, optionally wherein the plant matter comprises or consists of corn husks and/or cellulose; and/or
animal matter; and/or
fibrous materials; and/or
a bead;
optionally wherein the solid support comprises or consists of waste material.

5. The method of claim 3, wherein the solid support comprises or consists of a plant seed, optionally a viable plant seed.

6. The method of claim 5, wherein the Carbonic Anhydrase is immobilised to the seed coat, optionally wherein the immobilisation is carried out by spraying the seed with, or dipping the seed into, or otherwise coating the seed with, a liquid comprising the carbonic anhydrase.

7. The method of claim 6, wherein the solid support is immobilised to the seed coat before seed priming, during seed priming, or after seed priming.

8. The method of claim 5, wherein the seed is an agricultural crop species, optionally selected from:
a cereal, corn, barley, wheat, maize, millet, oats, rye, rice or sorghum;
a pulse- or bean-species such as soy;
or a vegetable crop such as potato, beet, carrot, sweet potato, turnip cabbage or lettuce.

9. The method of claim 5, wherein the seed is cotton, alfalfa, oilseed rape or a grass.

10. The method of claim 5, wherein the seed is a tree or shrub species.

11. The method of claim 3, wherein the solid support comprises or consists of a plant, optionally a spermatophyte.

12. The method of claim 11, wherein the Carbonic Anhydrase is immobilised to the plant stem and/or plant root, typically the plant root optionally prior to contacting the root with the soil.

13. The method of claim 3, wherein the solid support has a procurement cost of $20 or less per kg, $10 or less per kg, $5 or less per kg, $1 or less per kg, or $0.5 or less per kg.

14. The method of claim 2, wherein the Carbonic Anhydrase is immobilised to the rock, optionally directly to the rock, optionally by covalent bond or by non-covalent means such as physical adsorption.

15. The method of claim 1, wherein:
the Carbonic Anhydrase has a structural modification to the enzyme which is intended to increase retention at a specified depth and/or location in or on the soil, and/or
the Carbonic Anhydrase is provided in a mixture, formulation or composition which is intended to increase retention at a specified depth and/or location in or on the soil.

16. The method of claim 1, wherein the Carbonic Anhydrase is retained in or on top soil; optionally wherein the top soil extends 1 metre or less from the soil surface, optionally 50 centimetres or less, 30 centimetres or less, 25 centimetres or less, 20 centimetres or less, 15 centimetres or less, or 10 centimetres or less.

17. The method of claim 1, wherein the Carbonic Anhydrase is substantially retained at or around the roots of a plant, optionally in contact with one or more roots.

18. The method of claim 1, wherein
(i) the soil is within:
agricultural land, optionally wherein the agricultural land is arable land or pasture land;
waste land;
brownfield land; or
industrial land;
and/or
(ii) the applying step comprises spraying the Carbonic Anhydrase onto the soil, optionally wherein the Carbonic Anhydrase is present in a slurry when sprayed, optionally wherein the slurry comprises rock, and optionally wherein the slurry is sprayed using agricultural equipment for example a spray bar or broadcast spreader; and/or
(iii) the content of carbon retained in the soil increases as a result of the Carbonic Anhydrase having been applied to the soil.

19. The method of claim 2, wherein the Carbonic Anhydrase and rock are applied to the soil simultaneously, contemporaneously or at different times.

20. The method of claim 2, wherein the rock is a source of metal ions; optionally wherein the metal ions are cations, optionally wherein the cations are any of Mg+2 or Ca+2.

21. The method of claim 2, wherein:
the rock comprises silica, optionally wherein the rock comprising silica is basalt or olivine; and/or
the rock is particulate, optionally having a particle size diameter of 5 millimetres of less, or 4 millimetres or less, and/or having a 500 micrometres or greater, optionally wherein the rock particle size diameter is about 500 micrometres to about 10 millimetres, or is about 500 micrometres to about 4000 micrometres, or is about 500 micrometres to about 2000 micrometres.

22. The method of claim 2, wherein the method comprises the step of reducing the rock in size before application to the soil, optionally wherein the rock is reduced in size by crushing, grinding, sieving, milling, pulverisation, and/or ball milling.

23. The method of claim 2, wherein the rock is applied to the soil as a dust, powder, or particulate.

24. The method of claim 1, wherein the Carbonic Anhydrase comprises an animal carbonic anhydrase, a plant carbonic anhydrase, and/or a bacterial carbonic anhydrase; optionally wherein the animal Carbonic Anhydrase comprises bovine erythrocyte carbonic anhydrase;
optionally wherein the carbonic anhydrase is within a cell when it is applied to the soil.

25. The method of claim 1, wherein the Carbonic Anhydrase comprises an alpha, beta, delta, gamma, or zeta carbonic anhydrase.

26. The method of claim 1, wherein the Carbonic Anhydrase is mesophilic or cryophilic.

27. The method of claim 1, wherein the atmospheric carbon is sequestered as a carbonate, optionally as a magnesium carbonate or a calcium carbonate.

28. A sequestered carbon quantity obtained or obtainable by the method of claim 1.

29. An enzyme immobilised to a plant or plant seed, optionally wherein the enzyme is carbonic anhydrase.

30. An enzyme immobilised to a plant according to claim 29, wherein:
the enzyme is mammalian; and/or
the enzyme is obtained from non-human animal blood; and/or
the enzyme is obtained from bovine or ovine blood; and/or
the enzyme is immobilised to the plant root.

31. A method of sequestering atmospheric carbon comprising using the Carbonic Anhydrase according to claim 29.

32. A mixture comprising Carbonic Anhydrase and seed, optionally wherein the Carbonic Anhydrase is immobilised to the seed.

33. A mixture comprising Carbonic Anhydrase and soil, optionally wherein the Carbonic Anhydrase is immobilised to a solid support.

34. A mixture comprising Carbonic Anhydrase and rock, optionally wherein the Carbonic Anhydrase is immobilised to a solid support.

35. A mixture comprising carbonic anhydrase, rock and soil, optionally wherein the Carbonic Anhydrase is immobilised to a solid support.

36. A method of sequestering atmospheric carbon comprising using the mixture according to claim 32.

37. A method of sequestering atmospheric carbon using the mixture according to claim 32.

38. A method of applying an enzyme to a plant or plant seed, comprising dipping the plant or plant seed into, or spraying the plant or plant seed with, a liquid comprising the enzyme.

39. The method according to claim 38, wherein the enzyme is carbonic anhydrase.
